(19) United States
(12) Patent Application Publication
(10) Pub. No.: US 2011/0288084 A1
(43) Pub. Date: Nov. 24, 2011

(54) [4-( 1-AMINO-ETHYL) - CYCLOHEXYL ] - METHYL - AMINES AS ANTIBACTERIALS

(75) Inventors: Mark Cronin, Waltham, MA (US); Bolin Geng, Waltham, MA (US); Folkert Reck, Waltham, MA (US)

(73) Assignee: ASTRAZENECA AB, Sodertaljie (SE)

(21) Appl. No.: 13/128,596

(22) PCT Filed: Nov. 13, 2009
(86) PCT No.: PCT/GB09/51532

§ 371 Date: Aug. 10, 2011

(30) Foreign Application Priority Data
Nov. 14, 2008 (US) 61/114706
Mar. 26, 2009 (US) 61/163848

(51) Int. Cl. A61K 31/5383; A61P 31/04; C07D 498/04

(52) U.S. Cl. 514/230.5; 544/105

(57) ABSTRACT

The present invention relates to compounds of Formula (I) and pharmaceutically acceptable salts thereof, to their use in the treatment of bacterial infections, and to their methods of preparation.

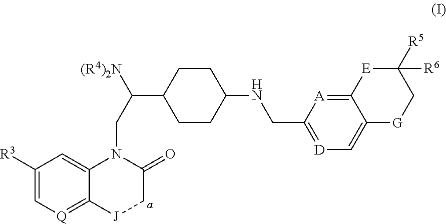

### FIELD OF INVENTION

The present invention relates to novel animocyclohexyl compounds, pharmaceutical compositions thereof, and methods of use. In addition, the present invention relates to therapeutic methods for the treatment of bacterial infections.

### BACKGROUND

The international health community continues to express serious concern that the evolution of antibacterial resistance will result in strains against which currently available antibacterial agents will be ineffective. For example, resistant strains of Gram-positive pathogens such as methicillin-resistant Staphylococcus aureus (MRSA), methicillin-resistant coagulase-negative staphylococci (MRCNS), penicillin-resistant Streptococcus pneumoniae and multiple resistant Enterococcus faecium are both difficult to treat and difficult to eradicate. Similarily, resistant strains of Gram-negative pathogens such as multi drug resistant Pseudomonas aeruginosa, Klebsiella pneumoniae and Acinetobacter baumannii are both difficult to treat and difficult to eradicate. Consequently, in order to overcome the threat of widespread multi-drug resistant organisms, there is an on-going need to develop new antibiotics, particularly those with either a novel mechanism of action and/or containing new pharmacophoric groups.

### SUMMARY

In accordance with the present invention, the applicants have hereby discovered compounds that possess the ability to act as antimicrobial agents.

The present invention provides compounds of Formula (I):

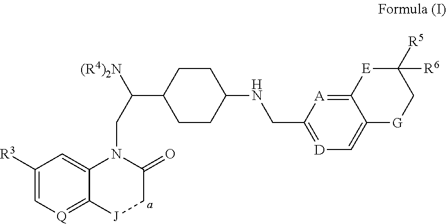

or pharmaceutically acceptable salts thereof.

Typical compounds of Formula (I) are believed to possess antibacterial activity, and are therefore believed to be useful for the treatment of bacterial infections. The present invention also provides processes for the preparation of compounds of Formula (I), pharmaceutical compositions containing them as the active ingredient, their use as medicaments, methods of using such compounds, and their use in the manufacture of medicaments for the treatment of bacterial infections in warm-blooded animals such as man.

It is expected that typical compounds of Formula (I) possess beneficial efficacious, metabolic, toxicological, and/or pharmacodynamic properties.

### DETAILED DESCRIPTION OF THE INVENTION

The present invention provides compounds of Formula (I):

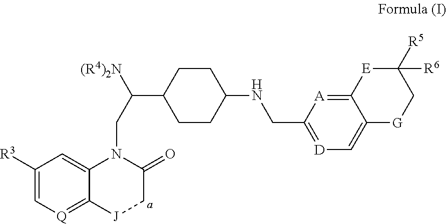

or pharmaceutically acceptable salts thereof, wherein
A is selected from CH and N;
D is selected from CH and N;
wherein at least one of A and D is nitrogen;
   E is selected from O, NH, and S,
wherein
i) E is NH if R⁵ and R⁶ together form ═O; and
ii) E is selected from O and S if R⁵ and R⁶ are each H;
   G is selected from O and S;
   the bond represented with a dashed line between J and carbon “a” is a single bond or double bond;
   J is selected from C—R¹, O, and N,
wherein
i) J is selected from C—R¹ and N if the bond connecting J and carbon “a” is a double bond; and
ii) J is O if the bond connecting J and carbon “a” is a single bond;
   Q is selected from C—R² and N;
   R¹ is selected from H, halo, —CN, C₁₋₆alkyl, C₂₋₆alkenyl, C₂₋₆alkynyl, carbocyclyl, heterocyclyl, —OR^{1a}, —SR^{1a}, —N(R^{1a})₂, —N(R^{1a})C(O)R^{1b}, —N(R^{1a})N(R^{1a})₂, —NO₂, —N(R^{1a})OR^{1a}, —ON(R^{1a})₂, —C(O)H, —C(O)R^{1b}, —C(O)₂R^{1a}, —C(O)N(R^{1a})₂, —C(O)N(R^{1a})(OR^{1a})—OC(O)N(R^{1a})₂, —N(R^{1a})C(O)₂R^{1a}, —N(R^{1a})C(O)N(R^{1a})₂, —OC(O)R^{1b}, —S(O)R^{1b}, —S(O)₂R^{1b}, —S(O)₂N(R^{1a})₂, —N(R^{1a})S(O)₂R^{1b}, —C(R^{1a})═N(R^{1a}), and —C(R^{1a})═N(OR^{1a}), wherein said C₁₋₆alkyl, C₂₋₆alkenyl, C₂₋₆alkynyl, carbocyclyl, and heterocyclyl are optionally substituted on carbon with one or more R¹⁰, and wherein any —NH— moiety of said heterocyclyl is optionally substituted with R¹⁰*;
   R^{1a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R¹⁰, and wherein if said heterocyclyl contains an —NH— moiety, that —NH— moiety is optionally substituted with R¹⁰*;
   R^{1b} in each occurrence is selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R¹⁰, and wherein if said heterocyclyl contains an —NH— moiety, that —NH— moiety is optionally substituted with R¹⁰*;
   R² is selected from H, halo, —CN, C₁₋₆alkyl, C₂₋₆alkenyl, C₂₋₆alkynyl, carbocyclyl, heterocyclyl, —OR^{2a}, —SR^{2a}, —N(R^{2a})₂, —N(R^{2a})C(O)R^{2b}, —N(R^{2a})N(R^{2a})₂, —NO₂, —N(R^{2a})OR^{2a}, —ON(R^{2a})₂, —C(O)H, —C(O)R^{2b}, —C(O)₂R^{2a}, —C(O)N(R^{2a})₂, —C(O)N(R^{2a})(OR^{2a})—OC(O)N(R^{2a})₂, —N(R^{2a})C(O)₂R^{2a}, —N(R^{2a})C(O)N(R^{2a})₂, —OC(O)R^{2b}, —S(O)R^{2b}, —S(O)₂R^{2b}, —S(O)₂N(R^{2a})₂, —N(R^{2a})S(O)₂R^{2b}, —C(R^{2a})═N(R^{2a}), and —C(R^{2a})═N(OR^{2a}), wherein said C₁₋₆alkyl, C₂₋₆alkenyl, C₂₋₆alkynyl, carbocyclyl, and heterocyclyl are optionally substituted on carbon with one or more R²⁰, and wherein any —NH— moiety of said heterocyclyl is optionally substituted with R²⁰*;
   R^{2a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R²⁰, and wherein if said heterocyclyl contains an —NH— moiety, that —NH— moiety is optionally substituted with R²⁰*;
   R^{2b} in each occurrence is selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R²⁰, and wherein if said heterocyclyl contains an —NH— moiety, that —NH— moiety is optionally substituted with R²⁰*;
   R³ is selected from H, halo, —CN, C₁₋₆alkyl, C₂₋₆alkenyl, C₂₋₆alkynyl, carbocyclyl, heterocyclyl, —OR^{3a}, —SR^{3a}, —N(R^{3a})₂, —N(R^{3a})C(O)R^{3b}, —N(R^{3a})N(R^{3a})₂, —NO₂, —N(R^{3a})(OR^{3a}), —O—N(R^{3a})₂, —C(O)H, —C(O)R^{3b}, —C(O)₂R^{3a}, —C(O)N(R^{3a})₂, —C(O)N(R^{3a})(OR^{3a}), —OC(O)N(R^{3a})₂, —N(R^{3a})C(O)₂R³, —N(R^{3a})C(O)N(R^{3a})₂, —OC(O)R^{3b}, —S(O)R^{3b}, —S(O)₂R^{3b}, —S(O)₂N(R^{3a})₂, —N(R^{3a})S(O)₂R^{3b}, —C(R^{3a})═N(R^{3a}), and —C(R^{3a})═N(OR^{3a}), wherein said C₁₋₆alkyl, C₂₋₆alkenyl, C₂₋₆alkynyl, carbocyclyl, and heterocyclyl are optionally substituted on carbon with one or more R³⁰, and wherein if said heterocyclyl contains an —NH— moiety, that —NH— moiety is optionally substituted with R³⁰*;
   R^{3a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R³⁰, and wherein if said heterocyclyl contains an —NH— moiety, that —NH— moiety is optionally substituted with R³⁰*;
   R^{3b} in each occurrence is selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R³⁰, and wherein if said heterocyclyl contains an —NH— moiety, that —NH— moiety is optionally substituted with R³⁰*;
   R⁴ in each occurrence is independently selected from H, C₁₋₆alkyl, carbocyclyl, heterocyclyl, —C(O)H, —C(O)R^{4b}, —C(O)₂R^{4a}, —C(O)N(R^{4a})₂, —S(O)R^{4b}, —S(O)₂R^{4b}, —S(O)₂N(R^{4a})₂, —C(R^{4a})═N(R^{4a}), and —C(R^{4a})═N(OR^{4a});
   R^{4a} in each occurrence is independently selected from H, C₁₋₆alkyl, carbocyclyl, and heterocyclyl, wherein said C₁₋₆alkyl, carbocyclyl, and heterocyclyl;
   R^{4b} in each occurrence is selected from C₁₋₆alkyl, C₂₋₆alkenyl, C₂₋₆alkynyl, carbocyclyl, and heterocyclyl;
   R⁵ and R⁶ are each hydrogen, or R⁵ and R⁶ together with the carbon to which they are attached form a —C(O)— group;
   R¹⁰ in each occurrence is independently selected from halo, —CN, C₁₋₆alkyl, C₂₋₆alkenyl, C₂₋₆alkynyl, carbocyclyl, heterocyclyl, —OR^{10a}, —SR^{10a}, —N(R^{10a})₂, —N(R^{10a})C(O)R^{10b}, —N(R^{10a})N(R^{10a})₂, —NO₂, —N(R^{10a})(OR^{10a}), —O—N(R^{10a})₂, —C(O)H, —C(O)R^{10b}, —C(O)₂R^{10a}, —C(O)N(R^{10a})₂, —C(O)N(R^{10a})(OR^{10a}), —OC(O)N(R^{10a})₂, —N(R^{10a})C(O)₂R^{10a}, —N(R^{10a})C(O)N(R^{10a})₂, —OC(O)R^{10b}, —S(O)R^{10b}, —S(O)₂R^{10b}, —S(O)₂N(R^{10a})₂, —N(R^{10a})S(O)₂R^{10b}, —C(R^{10a})═N(R^{10a}), and —C(R^{10a})═N(OR^{10a});
   R¹⁰* in each occurrence is independently selected from C₁₋₆alkyl, carbocyclyl, heterocyclyl, —C(O)H, —C(O)R^{10b}, —C(O)₂R^{10a}, —C(O)N(R^{10a})₂, —S(O)R^{10b}, —S(O)₂R^{10b}, —S(O)₂N(R^{10a})₂, —C(R^{10a})═N(R^{10a}), and —C(R^{10a})═N(OR^{10a});
   R^{10a} in each occurrence is independently selected from H, C₁₋₆alkyl, carbocyclyl, and heterocyclyl;
   R^{10b} in each occurrence is independently selected from C₁₋₆alkyl, C₂₋₆alkenyl, C₂₋₆alkynyl, carbocyclyl, and heterocyclyl;
   R²⁰ in each occurrence is independently selected from halo, —CN, C₁₋₆alkyl, C₂₋₆alkenyl, C₂₋₆alkynyl, carbocyclyl, heterocyclyl, —OR^{20a}, —SR^{20a}, —N(R^{20a})₂, —N(R^{20a})C(O)R^{20b}, —N(R^{20a})N(R^{20a})₂, —NO₂, —N(R^{20a})—OR^{20a}, —O—N(R^{20a})₂, —C(O)H, —C(O)R^{20b}, —C(O)₂R^{20a}, —C(O)N(R^{20a})₂, —C(O)N(R^{20a})(OR^{20a}), —OC(O)N(R^{20a})₂, —N(R^{20a})C(O)₂R^{20a}, —N(R^{20a})C(O)N(R^{20a})₂, —OC(O)R^{20b}, —S(O)R^{20b}, —S(O)₂R^{20b}, —S(O)₂N(R^{20a})₂, —N(R^{20a})S(O)₂R^{20b}, —C(R^{20a})═N(R^{20a}), and —C(R^{20a})═N(OR^{20a});
   R²⁰* in each occurrence is independently selected from —CN, C₁₋₆alkyl, carbocyclyl, heterocyclyl, —OR^{20a}, —N(R^{20a})₂, —C(O)H, —C(O)R^{20b}, —C(O)₂R^{20a}, —C(O)N(R^{20a})₂, —S(O)R^{20b}, —S(O)₂R^{20b}, —S(O)₂N(R^{20a})₂, —C(R^{20a})═N(R^{20a}), and —C(R^{20a})═N(OR^{20a});
   R^{20a} in each occurrence is independently selected from H, C₁₋₆alkyl, carbocyclyl, and heterocyclyl;
   R^{20b} in each occurrence is independently selected from C₁₋₆alkyl, C₂₋₆alkenyl, C₂₋₆alkynyl, carbocyclyl, and heterocyclyl;
   R³⁰ in each occurrence is independently selected from halo, —CN, C₁₋₆alkyl, C₂₋₆alkenyl, C₂₋₆alkynyl, carbocyclyl, heterocyclyl, —OR^{30a}, —SR^{30a}, —N(R^{30a})₂, —N(R^{30a})C(O)R^{30b}, —N(R^{30a})N(R^{30a})₂, —NO₂, —N(R^{30a})(OR^{30a}), —O—N(R^{30a})₂, —C(O)H, —C(O)R^{30b}, —C(O)₂R^{30a}, —C(O)N(R^{30a})₂, —C(O)N(R^{30a})(OR^{30a}), —OC(O)N(R^{30a})₂, —N(R^{30a})C(O)₂R^{30a}, —N(R^{30a})C(O)N(R^{30a})₂, —OC(O)R^{30b}, —S(O)R^{30b}, —S(O)₂R^{30b}, —S(O)₂N(R^{30a})₂, —N(R^{30a})S(O)₂R^{30b}, —C(R^{30a})═N(R^{30a}), and —C(R^{30a})═N(OR^{30a});
   R³⁰* in each occurrence is independently selected from —CN, C₁₋₆alkyl, carbocyclyl, heterocyclyl, —OR^{30a}, —N(R^{30a})₂, —C(O)H, —C(O)R^{30b}, —C(O)₂R^{30a}, —C(O)N(R^{30a})₂, —S(O)R^{30b}, —S(O)₂R^{30b}, —S(O)₂N(R^{30a})₂, —C(R^{30a})═N(R^{30a}), and —C(R^{30a})═N(OR^{30a});
   R^{30a} in each occurrence is independently selected from H, C₁₋₆alkyl, carbocyclyl, and heterocyclyl; and
   R^{30b} in each occurrence is independently selected from C₁₋₆alkyl, C₂₋₆alkenyl, C₂₋₆alkynyl, carbocyclyl, and heterocyclyl.

In this specification the prefix C_{x-y} as used in terms such as C_{x-y} alkyl and the like (where x and y are integers) indicates the numerical range of carbon atoms that are present in the group; for example, C₁₋₄alkyl includes C₁alkyl(methyl), C₂alkyl(ethyl), C₃alkyl(propyl and isopropyl) and C₄alkyl(butyl, 1-methylpropyl, 2-methylpropyl, and t-butyl).

Where a particular R group (e.g. R^{1a}, R¹⁰, etc.) is present in a compound of Formula (I) more than once, it is intended that each selection for that R group is independent at each occurrence of any selection at any other occurrence. For example, the —N(R)₂ group is intended to encompass: 1) those —N(R)₂ groups in which both R substituents are the same, such as those in which both R substituents are, for example, C₁₋₆alkyl; and 2) those —N(R)₂ groups in which each R substituent is different, such as those in which one R substituent is, for example, H, and the other R substituent is, for example, carbocyclyl.

Unless specifically stated, the bonding atom of a group may be any suitable atom of that group; for example, propyl includes prop-1-yl and prop-2-yl.

Alkyl—As used herein the term “alkyl” refers to both straight and branched chain saturated hydrocarbon radicals having the specified number of carbon atoms. References to individual alkyl groups such as “propyl” are specific for the straight chain version only and references to individual branched chain alkyl groups such as ‘isopropyl’ are specific for the branched chain version only. In one aspect, “C₁₋₆alkyl” may be methyl.

Alkenyl —As used herein, the term “alkenyl” refers to both straight and branched chain hydrocarbon radicals having the specified number of carbon atoms and containing at least one carbon-carbon double bond. For example, “C₂₋₆alkenyl” includes, but is not limited to, groups such as C₂₋₆alkenyl, C₂₋₄alkenyl, ethenyl, 2-propenyl, 2-methyl-2-propenyl, 3-butenyl, 4-pentenyl, and 5-hexenyl.

Alkynyl —As used herein, the term “alkynyl” refers to both straight and branched chain hydrocarbon radicals having the specified number of carbon atoms and containing at least one carbon-carbon triple bond. For example, “C₂₋₆alkynyl” includes, but is not limited to, groups such as C₂₋₆alkynyl, C₂₋₄alkynyl, ethynyl, 2-propynyl, 2-methyl-2-propynyl, 3-butynyl, 4-pentynyl, and 5-hexynyl.

Carbocyclyl —As used herein, the term “carbocyclyl” refers to a saturated, partially saturated, or unsaturated, mono or bicyclic carbon ring that contains 3 to 12 ring atoms, of which one or more —CH₂— groups may be optionally replaced with a corresponding number of —C(O)— groups. Illustrative examples of “carbocyclyl” include, but are not limited to, adamantyl, cyclopropyl, cyclobutyl, cyclopentyl, cyclopentenyl, cyclohexyl, cyclohexenyl, indanyl, naphthyl, oxocyclopentyl, 1-oxoindanyl, phenyl, and tetralinyl.

3- to 6-Membered Carbocyclyl—In one aspect, “carbocyclyl” may be “3- to 6-membered carbocyclyl.” As used herein, the term “3- to 6-membered carbocyclyl” refers to a saturated, partially saturated, or unsaturated monocyclic carbon ring containing 3 to 6 ring atoms, of which one or more —CH₂— groups may be optionally replaced with a corresponding number of —C(O)— groups. Illustrative examples of “3- to 6-membered carbocyclyl” include cyclopropyl, cyclobutyl, cyclopentyl, oxocyclopentyl, cyclopentenyl, cyclohexyl, and phenyl.

Halo —As used herein, the term “halo” includes fluoro, chloro, bromo and iodo. In one aspect, the term “halo” may refer to fluoro, chloro, and bromo. In another aspect, the term “halo” may refer to fluoro and chloro. In still another aspect, the term “halo” may refer to fluoro.

Heterocyclyl —As used herein, the term “heterocyclyl” refers to a saturated, partially saturated, or unsaturated, mono or bicyclic ring containing 4 to 12 ring atoms of which at least one ring atom is selected from nitrogen, sulfur, and oxygen, and which may, unless otherwise specified, be carbon or nitrogen linked, and of which a —CH₂— group can optionally be replaced by a —C(O)—. Ring sulfur atoms may be optionally oxidized to form S-oxides. Ring nitrogen atoms may be optionally oxidized to form N-oxides. Illustrative examples of the term “heterocyclyl” include, but are not limited to, 1,3-benzodioxolyl, 3,5-dioxopiperidinyl, furanyl, imidazolyl, indolyl, isoquinolinyl, isothiazolyl, isoxazolyl, morpholinyl, 2-oxa-5-azabicyclo[2.2.1]hept-5-yl, oxazolyl, 2-oxopyrrolidinyl, 2-oxo-1,3-thiazolidinyl, piperazinyl, piperidyl, 2H-pyranyl, pyrazolyl, pyridinyl, pyrrolyl, pyrrolidinyl, pyrrolidinyl, pyrimidinyl, pyrazinyl, pyrazolyl, pyridazinyl, 4-pyridonyl, quinolyl, tetrahydrofuranyl, tetrahydropyranyl, thiazolyl, thiadiazolyl, thiazolidinyl, thiomorpholinyl, thiophenyl, pyridine-N-oxidyl and quinoline-N-oxidyl.

5- or 6-Membered Heterocyclyl—In one aspect, “heterocyclyl” may be “5- or 6-membered heterocyclyl,” which refers to a saturated, partially saturated, or unsaturated, monocyclic ring containing 5 or 6 ring atoms, of which at least one ring atom is selected from nitrogen, sulfur, and oxygen, and of which a —CH₂— group may be optionally replaced by a —C(O)— group. Unless otherwise specified, “5- or 6-membered heterocyclyl” groups may be carbon or nitrogen linked. Ring nitrogen atoms may be optionally oxidized to form an N-oxide. Ring sulfur atoms may be optionally oxidized to form S-oxides. Illustrative examples of “5- or 6-membered heterocyclyl” include, but are not limited to, 3,5-dioxopiperidinyl, furanyl, imidazolyl, isothiazolyl, isoxazolyl, morpholino, oxazolyl, 2-oxopyrrolidinyl, 2-oxo-1,3-thiazolidinyl, piperazinyl, piperidyl, 2H-pyranyl, pyrazolyl, pyridinyl, pyrrolyl, pyrrolidinyl, pyrrolidinyl, pyrimidinyl, pyrazinyl, pyrazolyl, pyridazinyl, 4-pyridonyl, tetrahydrofuranyl, tetrahydropyranyl, thiazolyl, thiadiazolyl, thiazolidinyl, thiomorpholino, thioenyl, pyridine-N-oxidyl.

Effective Amount —As used herein, the phrase “effective amount” means an amount of a compound or composition which is sufficient enough to significantly and positively modify the symptoms and/or conditions to be treated (e.g., provide a positive clinical response). The effective amount of an active ingredient for use in a pharmaceutical composition will vary with the particular condition being treated, the severity of the condition, the duration of the treatment, the nature of concurrent therapy, the particular active ingredient(s) being employed, the particular pharmaceutically-acceptable excipient(s)/carrier(s) utilized, the route of administration, and like factors within the knowledge and expertise of the attending physician.

Leaving Group —As used herein, the phrase “leaving group” is intended to refer to groups readily displaceable by a nucleophile such as an amine nucleophile, and alcohol nucleophile, or a thiol nucleophile. Examples of suitable leaving groups include halo, such as chloro and bromo, and sulfonyloxy group, such as methanesulfonyloxy and toluene-4-sulfonyloxy.

Optionally substituted —As used herein, the phrase “optionally substituted,” indicates that substitution is optional and therefore it is possible for the designated group to be either substituted or unsubstituted. In the event a substitution is desired, any number of hydrogens on the designated group may be replaced with a selection from the indicated substituents, provided that the normal valency of the atoms on a particular substituent is not exceeded, and that the substitution results in a stable compound. Heterocyclyl groups containing nitrogen atoms may be substituted on ring carbon atoms and/or ring nitrogen atoms.

In one aspect, when a particular group is designated as being optionally substituted with one or more substituents, that particular group may be unsubstituted. In another aspect, the particular group may bear one substituent. In another aspect, the particular group may bear two substituents. In still another aspect, the particular group may bear three substituents. In yet another aspect, the particular group may bear four substituents. In a further aspect, the particular group may bear one or two substituents. In still a further aspect, the particular group may be unsubstituted, or may bear one or two substituents.

Pharmaceutically Acceptable—As used herein, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Protecting Group —As used herein, the term “protecting group” is intended to refer to those groups used to prevent selected reactive groups (such as carboxy, amino, hydroxy, and mercapto groups) from undergoing undesired reactions.

Illustrative examples of suitable protecting groups for a hydroxy group include, but are not limited to, an acyl group; alkanoyl groups such as acetyl; aroyl groups, such as benzoyl; silyl groups, such as trimethylsilyl; and arylmethyl groups, such as benzyl. The deprotection conditions for the above hydroxy protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively a silyl group such as trimethylsilyl may be removed, for example, by fluoride or by aqueous acid; or an arylmethyl group such as a benzyl group may be removed, for example, by hydrogenation in the presence of a catalyst such as palladium-on-carbon.

Illustrative examples of suitable protecting groups for an amino group include, but are not limited to, acyl groups; alkanoyl groups such as acetyl; alkoxycarbonyl groups, such as methoxycarbonyl, ethoxycarbonyl, and t-butoxycarbonyl; arylmethoxycarbonyl groups, such as benzyloxycarbonyl; and aroyl groups, such benzoyl. The deprotection conditions for the above amino protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulfuric, phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid, for example boron trichloride). A suitable alternative protecting group for a primary amino group is, for example, a phthaloyl group, which may be removed by treatment with an alkylamine, for example dimethylaminopropylamine or 2-hydroxyethylamine, or with hydrazine. Another suitable protecting group for an amine is, for example, a cyclic ether such as tetrahydrofuran, which may be removed by treatment with a suitable acid such as trifluoroacetic acid.

The protecting groups may be removed at any convenient stage in the synthesis using conventional techniques well known in the chemical art, or they may be removed during a later reaction step or during work-up.

Substantially Free—The phrase “substantially free” is intended to indicate that the specified entity is present in an amount less than 10%. In aspect, the specified entity is present in an amount less than 5%. In another aspect, the specified entity is present in an amount less than 2%. In still another aspect, the specified entity is present in an amount less than 1%. In yet another aspect, the specified entity is present in an amount less than 0.5%. In a further aspect, the specified entity is present in an amount less than 0.2%.

With reference to substituent R¹ for illustrative purposes, the following substituent definitions have the indicated meanings:

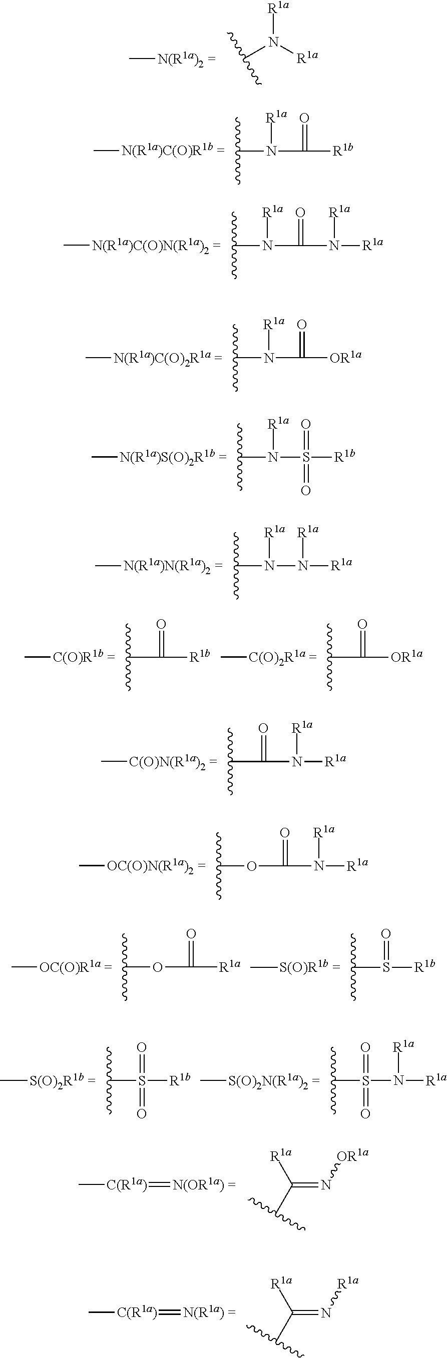

The structure shown for Formula (I) includes “carbon ‘a’.” This carbon atom is labelled with the letter “a” in Formula (I), and for the purposes of clarification is indicated below with a circle:

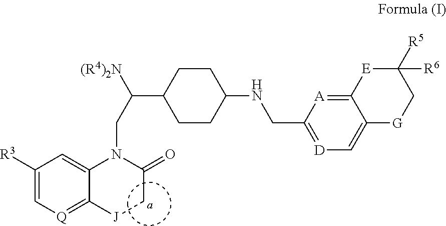

It is to be understood that the bond (represented in the structure with a dashed line) between J and carbon “a” is a single bond or double bond. For those instances in which the bond is a single bond, carbon “a” bears two hydrogens. For those instances in which the bond is a double bond, carbon “a” bears a single hydrogen.

In another aspect, the present invention relates to compounds of Formula (Ia), or pharmaceutically acceptable salts thereof, substantially free of the corresponding cis-isomer. It should be understood that for the purposes of discussing the compounds of Formula (Ia), the phrase “cis-isomer” refers to compounds of Formula (I) in which the two groups attached to the central cyclohexane ring are arranged in a relationship cis to each other.

The compounds discussed herein in many instances may have been named and/or checked with ACD/Name by ACD/Labs® and/or Electronic Lab Notebook by CambridgeSoft®.

Compounds of Formula (I) may form stable pharmaceutically acceptable acid or base salts, and in such cases administration of a compound as a salt may be appropriate. Examples of acid addition salts include acetate, adipate, ascorbate, benzoate, benzenesulfonate, bicarbonate, bisulfate, butyrate, camphorate, camphorsulfonate, choline, citrate, cyclohexyl sulfamate, diethylenediamine, ethanesulfonate, fumarate, glutamate, glycolate, hemisulfate, 2-hydroxyethyl-sulfonate, heptanoate, hexanoate, hydrochloride, hydrobromide, hydroiodide, hydroxymaleate, lactate, malate, maleate, methanesulfonate, meglumine, 2-naphthalenesulfonate, nitrate, oxalate, pamoate, persulfate, phenylacetate, phosphate, diphosphate, picrate, pivalate, propionate, quinate, salicylate, stearate, succinate, sulfamate, sulfanilate, sulfate, tartrate, tosylate (p-toluenesulfonate), trifluoroacetate, and undecanoate. Examples of base salts include ammonium salts; alkali metal salts such as sodium, lithium and potassium salts; alkaline earth metal salts such as aluminum, calcium and magnesium salts; salts with organic bases such as dicyclohexylamine salts and N-methyl-D-glucamine; and salts with amino acids such as arginine, lysine, ornithine, and so forth. Also, basic nitrogen-containing groups may be quaternized with such agents as: lower alkyl halides, such as methyl, ethyl, propyl, and butyl halides; dialkyl sulfates such as dimethyl, diethyl, dibutyl; diamyl sulfates; long chain halides such as decyl, lauryl, myristyl and stearyl halides; arylalkyl halides such as benzyl bromide and others. Non-toxic physiologically-acceptable salts are preferred, although other salts may be useful, such as in isolating or purifying the product.

The salts may be formed by conventional means, such as by reacting the free base form of the product with one or more equivalents of the appropriate acid in a solvent or medium in which the salt is insoluble, or in a solvent such as water, which is removed in vacuo or by freeze drying or by exchanging the anions of an existing salt for another anion on a suitable ion-exchange resin.

Compounds of Formula (I) have one or more chiral centres and/or geometric isomeric centres, and it is to be understood that the invention encompasses all such optical, diastereoisomers, and geometric isomers. The invention further relates to any and all tautomeric forms of the compounds of Formula (I).

When the prefix “trans” precedes the name of a compound, as in, for example, “trans-6-((4-(1-amino-2-(7-methoxy-2-oxo-1,5-naphthyridin-1(2H)-yl)ethyl)cyclohexylamino)methyl)-2H-pyrido[3,2-b][1,4]oxazin-3(4H)-one,” is to be understood that the “trans” designation refers to the relationship between the two substituents on the cyclohexane ring, indicated.

It is also to be understood that certain compounds of Formula (I) can exist in solvated as well as unsolvated forms such as, for example, hydrated forms. It is to be understood that the invention encompasses all such solvated forms.

It should be understood that the atoms of the compounds of Formula (I), and of any of the examples or embodiments disclosed herein, are intended to encompass all isotopes of the atoms. For example, H (or hydrogen) includes any isotopic form of hydrogen including ¹H, ²H (D), and ³H (T); C includes any isotopic form of carbon including ¹²C, ¹³C, and ¹⁴C; O includes any isotopic form of oxygen including ¹⁶O, ¹⁷O and ¹⁸O; N includes any isotopic form of nitrogen including ¹³N, ¹⁴N and ¹⁵N; P includes any isotopic form of phosphorous including ³¹P and ³²P; S includes any isotopic form of sulfur including ³²S and ³⁵S; F includes any isotopic form of fluorine including ¹⁹F and ¹⁸F; Cl includes any isotopic form of chlorine including ³⁵Cl, ³⁷Cl and ³⁶Cl; and the like. In one aspect, the compounds of Formula (I) include isotopes of the atoms covered therein in amounts corresponding to their naturally occurring abundance. However, in certain instances, it may be desirable to enrich one or more atom in a particular isotope which would normally be present in a lower abundance. For example, ¹H would normally be present in greater than 99.98% abundance; however, in one aspect, a compound of the invention may be enriched in ²H or ³H at one or more positions where H is present. In another aspect, when a compound of the invention is enriched in a radioactive isotope, for example ³H and ¹⁴C, the compound may be useful in drug and/or substrate tissue distribution assays. It is to be understood that the invention encompasses all such isotopic forms which are useful for treating bacterial infections.

Additional embodiments of the invention are as follows. These additional embodiments relate to compounds of Formula (I) and pharmaceutically acceptable salts thereof. Such specific substituents may be used, where appropriate, with any of the definitions, claims or embodiments defined hereinbefore or hereinafter.

In one aspect, the present invention relates to compounds of Formula (Ia):

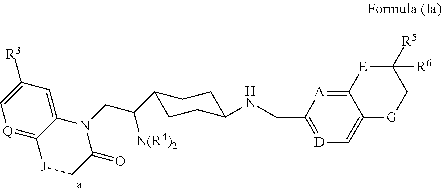

or pharmaceutically acceptable salts thereof, wherein A, D, E, G, J, Q, R³, R⁴, R⁵, R⁶, and the bond represented with a dashed line between J and carbon “a” are as defined hereinabove. For the sake of clarity, it is to be understood that in compounds of Formula (Ia), the groups on the cyclohexane ring are in a trans relationship to one another.

In another aspect, the present invention relates to compounds of Formula (Ia):

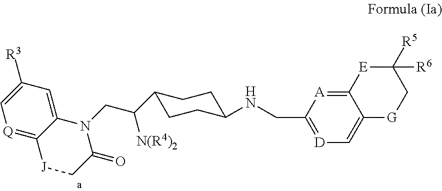

or pharmaceutically acceptable salts thereof, wherein

### A is N;

D is selected from CH and N;

### E is NH;

G is selected from O and S;
the bond represented with a dashed line between J and carbon “a” is a double bond;
J is selected from C—R¹ and N;
Q is selected from CH and N;
R¹ is selected from H and C₁₋₆alkyl;
R³ is selected from halo, —CN, and —OR^{3a};
R^{3a} is C₁₋₆alkyl;

### R⁴ is H; and

R⁵ and R⁶ together with the carbon to which they are attached form a —C(O)— group.

In still another aspect, the present invention relates to compounds of Formula (Ia):

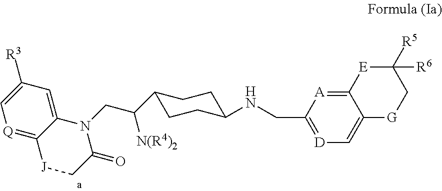

or pharmaceutically acceptable salts thereof, wherein

### A is N;

D is selected from CH and N;

### E is NH;

G is selected from O;
the bond represented with a dashed line between J and carbon “a” is a double bond;
J is selected from C—R¹ and N;
Q is selected from CH and N;
R¹ is selected from H and C₁₋₆alkyl;
R³ is selected from halo, —CN, and —OR^{3a};
R^{3a} is C₁₋₆alkyl;

### R⁴ is H; and

R⁵ and R⁶ together with the carbon to which they are attached form a —C(O)— group.

In yet another aspect, the present invention relates to compounds of Formula (Ia):

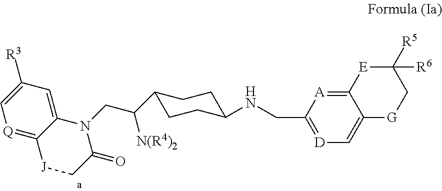

or pharmaceutically acceptable salts thereof, wherein

### A is N;

D is selected from CH and N;

### E is NH;

### G is O;

the bond represented with a dashed line between J and carbon “a” is a double bond;
J is selected from CH and N;
Q is selected from CH and N;
R³ is selected from —CN and —OR^{3a};
R^{3a} is C₁₋₆alkyl;

### R⁴ is H; and

R⁵ and R⁶ together with the carbon to which they are attached form a —C(O)— group.

In a further aspect, the present invention relates to compounds of Formula (Ia):

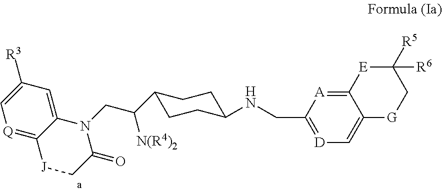

or pharmaceutically acceptable salts thereof, wherein

### A is N;

D is selected from CH and N;

### E is NH;

G is selected from O and S;
the bond represented with a dashed line between J and carbon “a” is a double bond;
J is selected from C—R¹ and N;
Q is selected from CH and N;
R¹ is selected from H and methyl;
R³ is selected from F, —CN, and —OMe;

### R⁴ is H; and

R⁵ and R⁶ together with the carbon to which they are attached form a —C(O)— group.

In still a further aspect, the present invention relates to compounds of Formula (Ia):

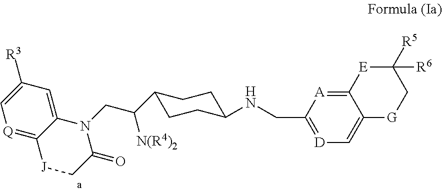

or pharmaceutically acceptable salts thereof, wherein

### A is N;

D is selected from CH and N;

### E is NH;

### G is O;

the bond represented with a dashed line between J and carbon “a” is a double bond;
J is selected from CH and N;
Q is selected from CH and N;
R³ is selected from —CN and methoxy;

### R⁴ is H; and

R⁵ and R⁶ together with the carbon to which they are attached form a —C(O)— group.

In one aspect, the present invention provides a compound of Formula (I) as illustrated by each of the Examples, free bases thereof, and pharmaceutically acceptable salts thereof, each of which provides a further independent aspect of the invention.

In a further aspect, the present invention provides a compound selected from:
6-((4-(1-Amino-2-(7-methoxy-2-oxo-1,5-naphthyridin-1(2H)-yl)ethyl)cyclohexylamino)methyl)-2H-pyrido[3,2-b][1,4]oxazin-3(4H)-one, trans enantiomer A;
6-((4-(1-Amino-2-(7-methoxy-2-oxo-1,5-naphthyridin-1(2H)-yl)ethyl)cyclohexylamino)methyl)-2H-pyrido[3,2-b][1,4]oxazin-3(4H)-one, trans enantiomer B;
6-((4-(1-Amino-2-(7-methoxy-2-oxoquinoxalin-1(2H)-yl)ethyl)cyclohexylamino)methyl)-2H-pyrido[3,2-b][1,4]oxazin-3(4H)-one, trans enantiomer A;
6-((4-(1-Amino-2-(7-methoxy-2-oxoquinoxalin-1(2H)-yl)ethyl)cyclohexylamino)methyl)-2H-pyrido[3,2-b][1,4]oxazin-3(4H)-one, trans enantiomer B;
2-((4-(1-Amino-2-(7-methoxy-2-oxoquinoxalin-1(2H)-yl)ethyl)cyclohexylamino)methyl)-6H-pyrimido[5,4-b][1,4]oxazin-7(8H)-one, trans enantiomer A;
2-((4-(1-Amino-2-(7-methoxy-2-oxoquinoxalin-1(2H)-yl)ethyl)cyclohexylamino)methyl)-6H-pyrimido[5,4-b][1,4]oxazin-7(8H)-one, trans enantiomer B;
1-(2-Amino-2-(4-((3-oxo-3,4-dihydro-2H-pyrido[3,2-b][1,4]oxazin-6-yl)methylamino)cyclohexyl)ethyl)-2-oxo-1,2-dihydroquinoline-7-carbonitrile, trans enantiomer A; and
1-(2-Amino-2-(4-((3-oxo-3,4-dihydro-2H-pyrido[3,2-b][1,4]oxazin-6-yl)methylamino)cyclohexyl)ethyl)-2-oxo-1,2-dihydroquinoline-7-carbonitrile, trans enantiomer B,
   or a pharmaceutically acceptable salt thereof.

In still a further aspect, the present invention provides a compound selected from:
trans-6-[({-4-[(1S)-1-amino-2-(7-methoxy-2-oxo-1,5-naphthyridin-1(2H)-yl)ethyl]cyclohexyl}amino)methyl]-2H-pyrido[3,2-b][1,4]oxazin-3 (4H)-one;
trans-6-[({4-[(1R)-1-amino-2-(7-methoxy-2-oxo-1,5-naphthyridin-1(2H)-yl)ethyl]cyclohexyl}amino)methyl]-2H-pyrido[3,2-b][1,4]oxazin-3(4H)-one;
trans-6-[({-4-[(1S)-1-amino-2-(7-methoxy-2-oxoquinoxalin-1(2H)-yl)ethyl]cyclohexyl}amino)methyl]-2H-pyrido[3,2-b][1,4]oxazin-3(4H)-one;
trans-6-[({4-[(1R)-1-amino-2-(7-methoxy-2-oxoquinoxalin-1(2H)-yl)ethyl]cyclohexyl}amino)methyl]-2H-pyrido[3,2-b][1,4]oxazin-3(4H)-one;
trans-2-[({-4-[(1S)-1-amino-2-(7-methoxy-2-oxoquinoxalin-1(2H)-yl)ethyl]cyclohexyl}amino)methyl]-6H-pyrimido[5,4-b][1,4]oxazin-7(8H)-one;
trans-2-[({4-[(1R)-1-amino-2-(7-methoxy-2-oxoquinoxalin-1(2H)-yl)ethyl]cyclohexyl}amino)methyl]-6H-pyrimido[5,4-b][1,4]oxazin-7(8H)-one;
trans-1-[(2S)-2-amino-2-(4-{[(3-oxo-3,4-dihydro-2H-pyrido[3,2-b][1,4]oxazin-6-yl)methyl]amino}cyclohexyl)ethyl]-2-oxo-1,2-dihydroquinoline-7-carbonitrile;
trans-1-[(2R)-2-amino-2-(4-{[(3-oxo-3,4-dihydro-2H-pyrido[3,2-b][1,4]oxazin-6-yl)methyl]amino}cyclohexyl)ethyl]-2-oxo-1,2-dihydroquinoline-7-carbonitrile;
trans-6-[({-4-[(1S)-1-amino-2-(7-fluoro-2-oxoquinoxalin-1(2H)-yl)ethyl]cyclohexyl}amino)methyl]-2H-pyrido[3,2-b][1,4]oxazin-3(4H)-one;
trans-6-[({4-[(1R)-1-amino-2-(7-fluoro-2-oxoquinoxalin-1(2H)-yl)ethyl]cyclohexyl}amino)methyl]-2H-pyrido[3,2-b][1,4]oxazin-3(4H)-one;
trans-2-[({-4-[(1S)-1-amino-2-(7-fluoro-2-oxoquinoxalin-1(2H)-yl)ethyl]cyclohexyl}amino)methyl]-6H-pyrimido[5,4-b][1,4]oxazin-7(8H)-one;
trans-2-[({4-[(1R)-1-amino-2-(7-fluoro-2-oxoquinoxalin-1(2H)-yl)ethyl]cyclohexyl}amino)methyl]-6H-pyrimido[5,4-b][1,4]oxazin-7(8H)-one;
trans-1-[(2S)-2-amino-2-(4-{[(3-oxo-3,4-dihydro-2H-pyrido[3,2-b][1,4]thiazin-6-yl)methyl]amino}cyclohexyl)ethyl]-2-oxo-1,2-dihydroquinoline-7-carbonitrile;
trans-1-[(2R)-2-amino-2-(4-{[(3-oxo-3,4-dihydro-2H-pyrido[3,2-b][1,4]thiazin-6-yl)methyl]amino}cyclohexyl)ethyl]-2-oxo-1,2-dihydroquinoline-7-carbonitrile;
trans-2-[({-4-[(1S)-1-amino-2-(7-methoxy-2-oxo-1,5-naphthyridin-1(2H)-yl)ethyl]cyclohexyl}amino)methyl]-6H-pyrimido[5,4-b][1,4]oxazin-7(8H)-one;
trans-2-[({4-[(1R)-1-amino-2-(7-methoxy-2-oxo-1,5-naphthyridin-1(2H)-yl)ethyl]cyclohexyl}amino)methyl]-6H-pyrimido[5,4-b][1,4]oxazin-7(8H)-one;
trans-6-{[(4-{(1S)-1-amino-2-[2-oxo-7-(phenylsulfanyl)-1,5-naphthyridin-1(2H)-yl]ethyl}cyclohexyl)amino]methyl}-2H-pyrido[3,2-b][1,4]oxazin-3(4H)-one;
trans-6-{[(4-{(1R)-1-amino-2-[2-oxo-7-(phenylsulfanyl)-1,5-naphthyridin-1(2H)-yl]ethyl}cyclohexyl)amino]methyl}-2H-pyrido[3,2-b][1,4]oxazin-3(4H)-one;
trans-6-[({4-[(1R)-1-amino-2-(7-fluoro-4-methyl-2-oxoquinolin-1(2H)-yl)ethyl]cyclohexyl}amino)methyl]-2H-pyrido[3,2-b][1,4]oxazin-3(4H)-one;
trans-6-[({4-[(1S)-1-amino-2-(7-fluoro-4-methyl-2-oxoquinolin-1(2H)-yl)ethyl]cyclohexyl}amino)methyl]-2H-pyrido[3,2-b][1,4]oxazin-3(4H)-one,
   or a pharmaceutically acceptable salt thereof.

### Biological Activity

The compounds of Formula (I) are of interest due to their antibacterial effects. The ability of the invention compounds disclosed herein to achieve an antibacterial effect may be evaluated with regard to their ability to inhibit the ParC enzyme of Escherichia coli using an assay based on the following protocol.

The assay utilizes the ATPase activity of the ParE subunit of reconstituted Escherichia coli ParC/ParE tetramer protein. Inhibition of ATPase activity may be monitored by reduced production of inorganic phosphate, a product of the ATPase reaction. Inorganic phosphate may be quantified using the ammonium molybdate/malachite green-based detection system. For determination of IC50 values, assays may be performed 384-well microtiter plates. Each well preferably contains a dilution range of the compound dissolved in DMSO. In addition, each well preferably contains: 20 mM Tris pH 8.0, 50 mM ammonium acetate, 0.16 mM ATP, 0.005% Brij-35, 8.0 mM magnesium chloride, 0.5 mM EDTA, 2.5% v/v glycerol, 5 mM dithiothreitol, 0.005 mg/mL sheared salmon sperm DNA, 0.5 nM E. coli ParC protein, 0.5 nM E. coli ParE protein. Final volume of assays is preferably 30 μL. Reactions may be incubated 24 hours at room temperature and then quenched with the addition of 45 μL malachite green reagent (Lanzetta, P. A., L. J. Alvarez, P. S. Reinach, and O. A. Candia (1979) Anal. Biochem. 100: 95-97) via a bulk reagent dispenser. Plates may be incubated 3-5 minutes at room temperature, and then absorbance at 650 nM may be measured using a Spectramax 384 plate reader.

When tested in an assay based on the one described above, the inhibitory activity of the following Examples was measured at the indicated IC₅₀. A hyphen indicates that an IC₅₀ measurement is not provided for that particular compound, and is not meant to imply that the particular compound does not possess IC₅₀ activity.

| | E. coli ParC |
|---|---|
| Example | IC₅₀ (μM) |
| 1 | 0.02 |
| 1(a) | 0.002 |
| 1(b) | 0.033 |
| 2 | — |
| 2(a) | 0.016 |
| 2(b) | 0.004 |
| 3 | — |
| 3(a) | 0.016 |
| 3(b) | 0.003 |
| 4 | 0.009 |
| 4(a) | 0.032 |
| 4(b) | 0.004 |
| 5 | 0.024 |
| 6 | 0.015 |
| 7 | 0.012 |
| 7(a) | — |
| 7(b) | 0.002 |
| 8 | 0.017 |
| 9 | 0.006 |
| 10 | 0.009 |

Thus, in one aspect there is provided a compound of Formula (I), or a pharmaceutically acceptable salt thereof, for use as a medicament.

It is expected that the compounds of the present invention will be useful in treating bacterial infections. In one aspect, the terms “infection” and “bacterial infection” may refer to a gynecological infection. In another aspect, the terms “infection” and “bacterial infection” may refer to a respiratory tract infection (RTI). In still another aspect, the terms “infection” and “bacterial infection” may refer to a sexually transmitted disease. In yet another aspect, the terms “infection” and “bacterial infection” may refer to a urinary tract infection. In a further aspect, the terms “infection” and “bacterial infection” may refer to acute exacerbation of chronic bronchitis (ACEB). In still a further aspect, the terms “infection” and “bacterial infection” may refer to acute otitis media. In yet a further aspect, the terms “infection” and “bacterial infection” may refer to acute sinusitis. In one aspect, the terms “infection” and “bacterial infection” may refer to an infection caused by drug resistant bacteria. In another aspect, the terms “infection” and “bacterial infection” may refer to catheter-related sepsis. In still another aspect, the terms “infection” and “bacterial infection” may refer to chancroid. In yet another aspect, the terms “infection” and “bacterial infection” may refer to chlamydia. In a further aspect, the terms “infection” and “bacterial infection” may refer to community-acquired pneumoniae (CAP). In still a further aspect, the terms “infection” and “bacterial infection” may refer to complicated skin and skin structure infection. In yet a further aspect, the terms “infection” and “bacterial infection” may refer to uncomplicated skin and skin structure infection. In one aspect, the terms “infection” and “bacterial infection” may refer to endocarditis. In another aspect, the terms “infection” and “bacterial infection” may refer to febrile neutropenia. In still another aspect, the terms “infection” and “bacterial infection” may refer to gonococcal cervicitis. In yet another aspect, the terms “infection” and “bacterial infection” may refer to gonococcal urethritis. In a further aspect, the terms “infection” and “bacterial infection” may refer to hospital-acquired pneumonia (HAP). In still a further aspect, the terms “infection” and “bacterial infection” may refer to osteomyelitis. In yet a further aspect, the terms “infection” and “bacterial infection” may refer to sepsis. In one aspect, the terms “infection” and “bacterial infection” may refer to syphilis.

In one aspect, the terms “infection” and “bacterial infection” may refer to an infection caused by Acinetobacter baumanii. In another aspect, the terms “infection” and “bacterial infection” may refer to an infection caused by Acinetobacter haemolyticus. In still another aspect, the terms “infection” and “bacterial infection” may refer to an infection caused by Acinetobacter junii. In yet another aspect, the terms “infection” and “bacterial infection” may refer to an infection caused by Acinetobacter johnsonii. In a further aspect, the terms “infection” and “bacterial infection” may refer to an infection caused by Acinetobacter lwoffi. In still a further aspect, the terms “infection” and “bacterial infection” may refer to an infection caused by Bacteroides bivius. In yet a further aspect, the terms “infection” and “bacterial infection” may refer to an infection caused by Bacteroides fragilis. In one aspect, the terms “infection” and “bacterial infection” may refer to an infection caused by Burkholderia cepacia. In another aspect, the terms “infection” and “bacterial infection” may refer to an infection caused by Campylobacter jejuni. In still another aspect, the terms “infection” and “bacterial infection” may refer to an infection caused by Chlamydia pneumoniae. In yet another aspect, the terms “infection” and “bacterial infection” may refer to an infection caused by Chlamydia urealyticus. In a further aspect, the terms “infection” and “bacterial infection” may refer to an infection caused by Chlamydophila pneumoniae. In still a further aspect, the terms “infection” and “bacterial infection” may refer to an infection caused by Clostridium difficili. In yet a further aspect, the terms “infection” and “bacterial infection” may refer to an infection caused by Enterobacter aerogenes. In one aspect, the terms “infection” and “bacterial infection” may refer to an infection caused by Enterobacter cloacae. In another aspect, the terms “infection” and “bacterial infection” may refer to an infection caused by Enterococcus faecalis. In still another aspect, the terms “infection” and “bacterial infection” may refer to an infection caused by Enterococcus faecium. In yet another aspect, the terms “infection” and “bacterial infection” may refer to an infection caused by Escherichia coli. In a further aspect, the terms “infection” and “bacterial infection” may refer to an infection caused by Gardnerella vaginalis. In still a further aspect, the terms “infection” and “bacterial infection” may refer to an infection caused by Haemophilus parainfluenzae. In yet a further aspect, the terms “infection” and “bacterial infection” may refer to an infection caused by Haemophilus influenzae. In one aspect, the terms “infection” and “bacterial infection” may refer to an infection caused by Helicobacter pylori. In another aspect, the terms “infection” and “bacterial infection” may refer to an infection caused by Klebsiella pneumoniae. In still another aspect, the terms “infection” and “bacterial infection” may refer to an infection caused by Legionella pneumophila. In yet another aspect, the terms “infection” and “bacterial infection” may refer to an infection caused by Methicillin-resistant Staphylococcus aureus. In a further aspect, the terms “infection” and “bacterial infection” may refer to an infection caused by Methicillin-susceptible Staphylococcus aureus. In still a further aspect, the terms “infection” and “bacterial infection” may refer to an infection caused by Moraxella catarrhalis. In yet a further aspect, the terms “infection” and “bacterial infection” may refer to an infection caused by Morganella morganii. In one aspect, the terms “infection” and “bacterial infection” may refer to an infection caused by Mycoplasma pneumoniae. In another aspect, the terms “infection” and “bacterial infection” may refer to an infection caused by Neisseria gonorrhoeae. In still another aspect, the terms “infection” and “bacterial infection” may refer to an infection caused by Penicillin-resistant Streptococcus pneumoniae. In yet another aspect, the terms “infection” and “bacterial infection” may refer to an infection caused by Penicillin-susceptible Streptococcus pneumoniae. In a further aspect, the terms “infection” and “bacterial infection” may refer to an infection caused by Peptostreptococcus magnus. In still a further aspect, the terms “infection” and “bacterial infection” may refer to an infection caused by Peptostreptococcus micros. In yet a further aspect, the terms “infection” and “bacterial infection” may refer to an infection caused by Peptostreptococcus anaerobius. In one aspect, the terms “infection” and “bacterial infection” may refer to an infection caused by Peptostreptococcus asaccharolyticus. In another aspect, the terms “infection” and “bacterial infection” may refer to an infection caused by Peptostreptococcus prevotii. In still another aspect, the terms “infection” and “bacterial infection” may refer to an infection caused by Peptostreptococcus tetradius. In yet another aspect, the terms “infection” and “bacterial infection” may refer to an infection caused by Peptostreptococcus vaginalis. In a further aspect, the terms “infection” and “bacterial infection” may refer to an infection caused by Proteus mirabilis. In still a further aspect, the terms “infection” and “bacterial infection” may refer to an infection caused by Pseudomonas aeruginosa. In yet a further aspect, the terms “infection” and “bacterial infection” may refer to an infection caused by Quinolone-Resistant Staphylococcus aureus. In one aspect, the terms “infection” and “bacterial infection” may refer to an infection caused by Quinolone-Resistant Staphylococcus epidermis. In another aspect, the terms “infection” and “bacterial infection” may refer to an infection caused by Salmonella typhi. In still another aspect, the terms “infection” and “bacterial infection” may refer to an infection caused by Salmonella paratyphi. In yet another aspect, the terms “infection” and “bacterial infection” may refer to an infection caused by Salmonella enteritidis. In a further aspect, the terms “infection” and “bacterial infection” may refer to an infection caused by Salmonella typhimurium. In still a further aspect, the terms “infection” and “bacterial infection” may refer to an infection caused by Serratia marcescens. In yet a further aspect, the terms “infection” and “bacterial infection” may refer to an infection caused by Staphylococcus aureus. In one aspect, the terms “infection” and “bacterial infection” may refer to an infection caused by Staphylococcus epidermidis. In another aspect, the terms “infection” and “bacterial infection” may refer to an infection caused by Staphylococcus saprophyticus. In still another aspect, the terms “infection” and “bacterial infection” may refer to an infection caused by Streptoccocus agalactiae. In yet another aspect, the terms “infection” and “bacterial infection” may refer to an infection caused by Streptococcus agalactiae. In a further aspect, the terms “infection” and “bacterial infection” may refer to an infection caused by Streptococcus pneumoniae. In still a further aspect, the terms “infection” and “bacterial infection” may refer to an infection caused by Streptococcus pyogenes. In yet a further aspect, the terms “infection” and “bacterial infection” may refer to an infection caused by Stenotrophomonas maltophilia. In one aspect, the terms “infection” and “bacterial infection” may refer to an infection caused by Ureaplasma urealyticum. In another aspect, the terms “infection” and “bacterial infection” may refer to an infection caused by Vancomycin-Resistant Enterococcus faecium. In still another aspect, the terms “infection” and “bacterial infection” may refer to an infection caused by Vancomycin-Resistant Enterococcus faecalis. In yet another aspect, the terms “infection” and “bacterial infection” may refer to an infection caused by Vancomycin-Resistant Staphylococcus aureus. In a further aspect, the terms “infection” and “bacterial infection” may refer to an infection caused by Vancomycin-Resistant Staphylococcus epidermis.

In one aspect, the terms “infection” and “bacterial infection” may refer to an infection caused by Acinetobacter spp. In another aspect, the terms “infection” and “bacterial infection” may refer to an infection caused by Bacteroides spp. In still another aspect, the terms “infection” and “bacterial infection” may refer to an infection caused by Burkholderia spp. In yet another aspect, the terms “infection” and “bacterial infection” may refer to an infection caused by Campylobacter spp. In a further aspect, the terms “infection” and “bacterial infection” may refer to an infection caused by Chlamydia spp. In still a further aspect, the terms “infection” and “bacterial infection” may refer to an infection caused by Chlamydophila spp. In yet a further aspect, the terms “infection” and “bacterial infection” may refer to an infection caused by Clostridium spp. In one aspect, the terms “infection” and “bacterial infection” may refer to an infection caused by Enterobacter spp. In another aspect, the terms “infection” and “bacterial infection” may refer to an infection caused by Enterococcus spp. In still another aspect, the terms “infection” and “bacterial infection” may refer to an infection caused by Escherichia spp. In yet another aspect, the terms “infection” and “bacterial infection” may refer to an infection caused by Gardnerella spp. In one aspect, the terms “infection” and “bacterial infection” may refer to an infection caused by Haemophilus spp. In another aspect, the terms “infection” and “bacterial infection” may refer to an infection caused by Helicobacter spp. In still another aspect, the terms “infection” and “bacterial infection” may refer to an infection caused by Klebsiella spp. In yet another aspect, the terms “infection” and “bacterial infection” may refer to an infection caused by Legionella spp. In one aspect, the terms “infection” and “bacterial infection” may refer to an infection caused by Moraxella spp. In another aspect, the terms “infection” and “bacterial infection” may refer to an infection caused by Morganella spp. In still another aspect, the terms “infection” and “bacterial infection” may refer to an infection caused by Mycoplasma spp. In yet another aspect, the terms “infection” and “bacterial infection” may refer to an infection caused by Neisseria spp. In a further aspect, the terms “infection” and “bacterial infection” may refer to an infection caused by Peptostreptococcus spp. In still a further aspect, the terms “infection” and “bacterial infection” may refer to an infection caused by Proteus spp. In yet a further aspect, the terms “infection” and “bacterial infection” may refer to an infection caused by Pseudomonas spp. In one aspect, the terms “infection” and “bacterial infection” may refer to an infection caused by Salmonella spp. In another aspect, the terms “infection” and “bacterial infection” may refer to an infection caused by Serratia spp. In still another aspect, the terms “infection” and “bacterial infection” may refer to an infection caused by Staphylococcus spp. In yet another aspect, the terms “infection” and “bacterial infection” may refer to an infection caused by Streptoccocus spp. In one aspect, the terms “infection” and “bacterial infection” may refer to an infection caused by Stenotrophomonas spp. In another aspect, the terms “infection” and “bacterial infection” may refer to an infection caused by Ureaplasma spp. In still another aspect, the terms “infection” and “bacterial infection” may refer to an infection caused by aerobes. In yet another aspect, the terms “infection” and “bacterial infection” may refer to an infection caused by obligate anaerobes. In one aspect, the terms “infection” and “bacterial infection” may refer to an infection caused by facultative anaerobes. In another aspect, the terms “infection” and “bacterial infection” may refer to an infection caused by gram-positive bacteria. In still another aspect, the terms “infection” and “bacterial infection” may refer to an infection caused by gram-negative bacteria. In yet another aspect, the terms “infection” and “bacterial infection” may refer to an infection caused by gram-variable bacteria. In one aspect, the terms “infection” and “bacterial infection” may refer to an infection caused by atypical respiratory pathogens.

Accordingly, in one aspect, there is provided the use of a compound of Formula (I), or a pharmaceutically acceptable salt thereof, in the manufacture of a medicament for the treatment of a bacterial infection in a warm-blooded animal such as man.

In another aspect, there is provided the use of a compound of Formula (I), or a pharmaceutically acceptable salt thereof, in the manufacture of a medicament for the production of an anti-bacterial effect in a warm-blooded animal such as man.

In still another aspect, there is provided a method for treating a bacterial infection in a warm-blooded animal such as man, said method including administering to said animal an effective amount of a compound of formula (I), or a pharmaceutically acceptable salt thereof.

In yet another aspect, there is provided a method for producing an anti-bacterial effect in a warm-blooded animal such as man, said method including administering to said animal an effective amount of a compound of formula (I), or a pharmaceutically acceptable salt thereof.

In a further aspect, there is provided a compound of Formula (I), or a pharmaceutically acceptable salt thereof, for use in treating a bacterial infection in a warm-blooded animal, such as man.

In still a further aspect, there is provided a compound of Formula (I), or a pharmaceutically acceptable salt thereof, for use in the production of an anti-bacterial effect in a warm-blooded animal, such as man.

A compound of Formula (I), or a pharmaceutically-acceptable salt thereof, for the therapeutic (including prophylactic) treatment of mammals including humans, in particular in treating infection, is normally formulated in accordance with standard pharmaceutical practice as a pharmaceutical composition.

Accordingly, in one aspect, there is provided a pharmaceutical composition including a compound of Formula (I), or a pharmaceutically acceptable salt thereof, and at least one pharmaceutically acceptable carrier, diluent, or excipient.

In another aspect, there is provided the use of a pharmaceutical composition including a compound of Formula (I), or a pharmaceutically acceptable salt thereof, in the manufacture of a medicament for the treatment of a bacterial infection in a warm-blooded animal such as man.

In still another aspect, there is provided the use of a pharmaceutical composition including a compound of Formula (I), or a pharmaceutically acceptable salt thereof, in the manufacture of a medicament for the production of an anti-bacterial effect in a warm-blooded animal such as man.

In yet another aspect, there is provided a method for treating a bacterial infection in a warm-blooded animal such as man, said method including administering to said animal an effective amount of a pharmaceutical composition including a compound of Formula (I), or a pharmaceutically acceptable salt thereof.

In a further aspect, there is provided a method for producing an anti-bacterial effect in a warm-blooded animal such as man, said method including administering to said animal an effective amount of a pharmaceutical composition including a compound of Formula (I), or a pharmaceutically acceptable salt thereof.

In still a further aspect, there is provided a pharmaceutical composition including a compound of Formula (I), or a pharmaceutically acceptable salt thereof, for use in treating a bacterial infection in a warm-blooded animal, such as man.

In yet a further aspect, there is provided a pharmaceutical composition including a compound of Formula (I), or a pharmaceutically acceptable salt thereof, for use in the production of an anti-bacterial effect in a warm-blooded animal, such as man.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing).

The compositions of the invention may be obtained by conventional procedures using conventional pharmaceutical excipients well known in the art. Thus, compositions intended for oral use may contain, for example, one or more coloring, sweetening, flavoring and/or preservative agents.

Suitable pharmaceutically acceptable excipients for a tablet formulation include, for example, inert diluents such as lactose, sodium carbonate, calcium phosphate or calcium carbonate; granulating and disintegrating agents such as corn starch or algenic acid; binding agents such as starch; lubricating agents such as magnesium stearate, stearic acid or talc; preservative agents such as ethyl or propyl p-hydroxybenzoate; and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form or in the form of nano or micronized particles together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives such as ethyl or propyl p-hydroxybenzoate; anti-oxidants such as ascorbic acid); coloring agents; flavoring agents; and/or sweetening agents such as sucrose, saccharine or aspartame.

Oily suspensions may be formulated by suspending the active ingredient in a vegetable oil such as arachis oil, olive oil, sesame oil or coconut oil or in a mineral oil such as liquid paraffin. The oily suspensions may also contain a thickening agent such as beeswax, hard paraffin or cetyl alcohol. Sweetening agents such as those set out above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavoring and coloring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavoring and preservative agents.

Syrups and elixirs may be formulated with sweetening agents such as glycerol, propylene glycol, sorbitol, aspartame or sucrose, and may also contain a demulcent, preservative, flavoring and/or coloring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Compositions for administration by inhalation may be in the form of a conventional pressurized aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 4 g of active agent compounded with an appropriate and convenient amount of excipients which may vary from about 5 to about 98 percent by weight of the total composition. Dosage unit forms will generally contain about 1 mg to about 500 mg of an active ingredient. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

As stated above the size of the dose required for the therapeutic or prophylactic treatment of a particular disease state will necessarily be varied depending on the host treated, the route of administration and the severity of the illness being treated. Preferably a daily dose in the range of 1-50 mg/kg is employed. Accordingly, the optimum dosage may be determined by the practitioner who is treating any particular patient.

In any of the pharmaceutical compositions, processes, methods, uses, medicaments, and manufacturing features mentioned herein, any of the alternate aspects of the compounds of the invention described herein also apply.

### Combinations

The compounds of the invention described herein may be applied as a sole therapy or may involve, in addition to a compound of the invention, one or more other substances and/or treatments. Such conjoint treatment may be achieved by way of the simultaneous, sequential or separate administration of the individual components of the treatment. Where the administration is sequential or separate, the delay in administering the second component should not be such as to lose the beneficial effect of the combination. Suitable classes and substances may be selected from one or more of the following:
i) other antibacterial agents for example macrolides e.g. erythromycin, azithromycin or clarithromycin; quinolones e.g. ciprofloxacin or levofloxacin; β-lactams e.g. penicillins e.g. amoxicillin or piperacillin; cephalosporins e.g. ceftriaxone or ceftazidime; carbapenems, e.g. meropenem or imipenem etc; aminoglycosides e.g. gentamicin or tobramycin; or oxazolidinones; and/or
ii) anti-infective agents for example, an antifungal triazole e.g. or amphotericin; and/or
iii) biological protein therapeutics for example antibodies, cytokines, bactericidal/permeability-increasing protein (BPI) products; and/or
iv) efflux pump inhibitors.

Therefore, in a further aspect of the invention there is provided a compound of Formula (I), or a pharmaceutically acceptable salt thereof and a chemotherapeutic agent selected from:
i) one or more additional antibacterial agents; and/or
ii) one or more anti-infective agents; and/or
iii) biological protein therapeutics for example antibodies, cytokines, bactericidal/permeability-increasing protein (BPI) products; and/or
iv) one or more efflux pump inhibitors.

### Process

If not commercially available, the necessary starting materials for the procedures such as those described herein may be made by procedures which are selected from standard organic chemical techniques, techniques which are analogous to the synthesis of known, structurally similar compounds, or techniques which are analogous to the described procedure or the procedures described in the Examples.

It is noted that many of the starting materials for synthetic methods as described herein are commercially available and/or widely reported in the scientific literature, or could be made from commercially available compounds using adaptations of processes reported in the scientific literature. The reader is further referred to Advanced Organic Chemistry, 5^{th} Edition, by Jerry March and Michael Smith, published by John Wiley & Sons 2001, for general guidance on reaction conditions and reagents.

It will also be appreciated that in some of the reactions mentioned herein it may be necessary/desirable to protect any sensitive groups in compounds. The instances where protection is necessary or desirable are known to those skilled in the art, as are suitable methods for such protection. Conventional protecting groups may be used in accordance with standard practice (for illustration see T. W. Greene, Protective Groups in Organic Synthesis, published by John Wiley and Sons, 1991) and as described hereinabove.

Compounds of Formula (I) may be prepared in a variety of ways. The processes and Examples shown below illustrate some methods useful for the synthesis of compounds of Formula (I) and intermediates which may be used for the synthesis of compounds of Formula (I) (wherein A, D, E, G, J, Q, R³, R⁴, R⁵, R⁶, and the bond represented with a dashed line between J and carbon “a”, unless otherwise defined, are as defined hereinabove; and wherein PG denotes a protecting group). Where a particular solvent or reagent is shown or referred to in the accompanying text, it is to be understood that the chemist of ordinary skill in the art will be able to modify and/or replace that solvent or reagent as necessary. The processes and Examples are not intended to present an exhaustive list of methods for preparing the compounds of Formula (I); rather, additional techniques of which the skilled chemist is aware of may be also be used for the compounds' synthesis. For example, various techniques described in PCT Pub. No. WO09/001,126 may be useful for the synthesis of the Examples herein. The claims are not intended to be limited to the structures shown in the Processes and Examples.

The skilled chemist will be able to use and adapt the information contained and referenced within the above references, and accompanying Examples therein and also the Examples herein, to obtain the necessary starting materials and products.

Process A—In one aspect, the present invention provides a process for preparing compounds of Formula (I), and pharmaceutically acceptable salts thereof, the process including reacting a compound of Formula (A):

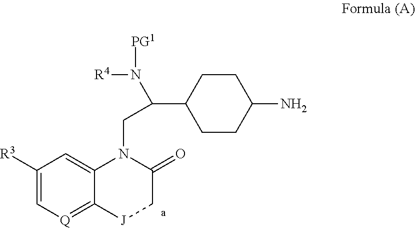

with a compound of Formula (B):

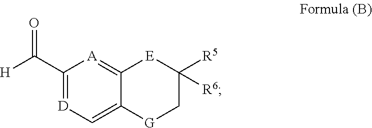

and thereafter if appropriate:
i) converting a compound of Formula (I) into another compound of Formula (I);
ii) removing any protecting groups; and/or
iii) forming a pharmaceutically acceptable salt.

The reaction shown in Process A may be performed under standard reductive amination conditions. The reaction will beneficially be performed n the presence of a suitable drying agent (such as molecular sieves). Reducing agents suitable for the reductive amination include reducing agents such as sodium borohydride. Suitable protecting groups PG¹ include sulfonamide protecting groups such as tosyl and nosyl protecting groups.

Compounds of Formula (A) may be prepared from compounds of Formula (C):

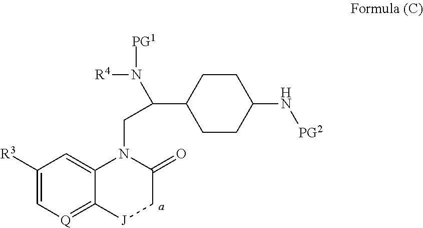

by removal of protecting group PG². In one aspect, there is provided a process for preparing compounds of Formula (C), the process including reacting a compound of Formula (D):

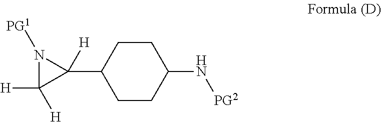

with a compound of Formula (E):

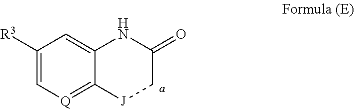

in the presence of a suitable base, and thereafter if appropriate:
i) converting a compound of Formula (I) into another compound of Formula (I);
ii) removing any protecting groups; and/or
iii) forming a pharmaceutically acceptable salt,
   wherein PG¹ and PG² are protecting groups.

The reaction shown in the process above may be performed under standard nucleophilic substitution conditions. Suitable bases include bases such as sodium hydride. Suitable protecting groups PG¹ include sulfonamide protecting groups such as tosyl and nosyl protecting groups. Suitable protecting groups PG² include carbamate protecting groups such as t-butyl carbamate.

When an optically active form of a compound of the invention is required, it may be obtained by carrying out one of the above procedures using a pure enantiomer as a starting material, or by resolution of a mixture of the enantiomers or diastereomers of the final products or chiral intermediates using a standard procedure. The resolution of enantiomers may be achieved by chromatography on a chiral stationary phase, such as a Chiralpak® AD column. Consideration has to be given to solubility as well as resolution. Alternatively, resolution may be obtained by preparation and selective crystallization of a diastereomeric salt of a chiral intermediate or chiral product with a chiral acid, such as camphersulfonic acid. Alternatively, a method of stereoselective synthesis may be employed, for example by using a chiral variant of a protection group, a chiral catalyst or a chiral reagent where appropriate in the reaction sequence. Enzymatic techniques may also be useful for the preparation of optically active compounds and/or intermediates.

### EXAMPLES

The invention is now illustrated by, but not limited to, the following Examples, for which, unless otherwise stated:
(i) evaporations were carried out by rotary evaporation in vacuo and work-up procedures were carried out after removal of residual solids by filtration;
(ii) temperatures are quoted as ° C.; operations were carried out at room temperature, that is typically in the range 18-26° C. and without the exclusion of air unless otherwise stated, or unless the skilled person would otherwise work under an inert atmosphere;
(iii) column chromatography (by the flash procedure) was used to purify compounds and was performed on Merck Kieselgel silica (Art. 9385) unless otherwise stated;
(iv) in general, the course of reactions was followed by TLC, HPLC, or LC/MS and reaction times are given for illustration only; yields are given for illustration only and are not necessarily the maximum attainable;
(v) the structure of the end-products of the invention was generally confirmed by NMR and mass spectral techniques. Proton magnetic resonance spectra were generally determined in DMSO-d₆ unless otherwise stated, using a Bruker DRX-300 spectrometer or a Bruker DRX-400 spectrometer, operating at a field strength of 300 MHz, or 400 MHz, respectively. In cases where the NMR spectrum is complex, only diagnostic signals are reported. Chemical shifts are reported in parts per million downfield from tetramethylsilane as an external standard (δ scale) and peak multiplicities are shown thus: s, singlet; d, doublet; dd, doublet of doublets; dt, doublet of triplets; dm, doublet of multiplets; t, triplet, m, multiplet; br, broad. Fast-atom bombardment (FAB) mass spectral data were generally obtained using a Platform spectrometer (supplied by Micromass) run in electrospray and, where appropriate, either positive ion data or negative ion data were collected or using Agilent 1100 series LC/MS equipped with Sedex 75ELSD, and where appropriate, either positive ion data or negative ion data were collected. The lowest mass major ion is reported for molecules where isotope splitting results in multiple mass spectral peaks (for example when chlorine is present). Reverse Phase HPLC was carried out using YMC Pack ODS-AQ (100×20 mmID, S-5μ particle size, 12 nm pore size) on Agilent instruments;
(vi) each intermediate was purified to the standard required for the subsequent stage and was characterized in sufficient detail to confirm that the assigned structure was correct; purity was assessed by HPLC, TLC, or NMR and identity was determined by infra-red spectroscopy (IR), mass spectroscopy or NMR spectroscopy as appropriate; and
(vii) the following abbreviations may be used:
   TLC is thin layer chromatography; HPLC is high pressure liquid chromatography; MPLC is medium pressure liquid chromatography; NMR is nuclear magnetic resonance spectroscopy; DMSO is dimethylsulfoxide; CDCl₃ is deuterated chloroform; MeOD is deuterated methanol, i.e. D₃COD; MS is mass spectroscopy; ESP (or ES) is electrospray; EI is electron impact; APCI is atmospheric pressure chemical ionization; THF is tetrahydrofuran; DCM is dichloromethane; MeOH is methanol; DMF is dimethylformamide; EtOAc is ethyl acetate; LC/MS is liquid chromatography/mass spectrometry; h is hour(s); min is minute(s); d is day(s); MTBD is N-methyl-1,5,7-triazabicyclo[4.4.0]dec-5-ene; TFA is trifluoroacetic acid; v/v is ratio of volume/volume; Boc denotes t-butoxycarbonyl; Cbz denotes benzyloxycarbonyl; Bz denotes benzoyl; atm denotes atmospheric pressure; rt denotes room temperature; mg denotes milligram; g denotes gram; μL denotes microliter; mL denotes milliliter; L denotes liter; μM denotes micromolar; mM denotes millimolar; M denotes molar; N denotes normal; nm denotes nanometer.

### Intermediate 1

### Trans-tert-butyl 4-(oxiran-2-yl)cyclohexylcarbamate

To a solution of trans-tert-butyl 4-vinylcyclohexylcarbamate (1.966 g, 8.73 mmol) in dichloromethane (20 mL) was added m-CPBA (2.25 g, 13.04 mmol) at room temperature. The reaction mixture was stirred at room temperature for 6 hours and then partitioned between ethyl acetate and saturated aqueous sodium carbonate solution. The organic layer was dried over magnesium sulfate and concentrated under reduced pressure to give the product as a colourless solid, 1.955 g (93%).

¹H NMR (DMSO-d₆) δ: 6.72 (d, br, 1H); 3.30 (s, br, 1H); 2.64 (m, 3H); 1.78 (m, 3H); 1.62 (s, br, 1H); 1.37 (s, 9H); 1.10 (m, 5H).

### Intermediate 2

### Trans-tert-butyl (2-(4-((tert-butoxycarbonyl)amino)cyclohexyl)-2-hydroxyethyl)((2-nitrophenyl)sulfonyl)carbamate

To a solution of trans-tert-butyl 4-(oxiran-2-yl)cyclohexylcarbamate (Intermediate 1, 1.10 g, 4.56 mmol) in THF (20 mL) was added Jacobsen Co(III) (salen) acetate catalyst (prepared through one-electron oxidation of (S,S)-(+)-N,N′-bis(3,5-di-t-butylsalicylidene)-1,2-cyclohexanediaminocobalt(II) in open air in acetic acid, Reference: S. Schaus, et al. J. Amer. Chem. Soc. 2002, vol. 124, 1307) (55 mg, 0.08 mmol, prepared according to literature procedure). The mixture was stirred at room temperature for three hours and then tert-butyl 2-nitrophenylsulfonylcarbamate (688 mg, 2.28 mmol) was added. The mixture was stirred over night and then more tert-butyl 2-nitrophenylsulfonylcarbamate (688 mg, 2.28 mmol) was added and stirring was continued. THF was removed under reduced pressure and the residue was purified by chromatography on silica gel with 30% ethyl acetate in hexanes to give the product as a light pink solid, 1.93 g (78%).

MS (ES): 566 (MNa⁺) for C₂₄H₃₇N₃O₉S

¹H NMR (DMSO-d₆) δ: 7.6-8.6 (m, 5H); 6.71 (d, br, 1H); 4.93 (d, 1H); 4.02 (t, 1H); 3.68 (d 1H); 3.50 (s, br, 1H); 3.15 (s, br, 1H); 1.78 (m, 3H); 1.81 (d, 2H); 1.61 (m, 1H); 1.37 (s, 9H); 1.21 (s, 9H); 1.14 (m, 2H).

### Intermediate 3

### Trans-2,2,2-trifluoro-N-(4-(1-hydroxy-2-(2-nitrophenylsulfonamido)ethyl)cyclohexyl)acetamide

To a solution of trans-tert-butyl (2-(4-((tert-butoxycarbonyl)amino)cyclohexyl)-2-hydroxyethyl)((2-nitrophenyl)sulfonyl)carbamate (Intermediate 2, 1.93 g, 3.55 mmol) in dichloromethane (5 mL) was added trifluoroacetic acid (TFA) (4 mL, 51.92 mmol) in dichloromethane (4 mL). The mixture was stirred at room temperature for 12 hours. More TFA (4 mL) was added and the mixture was heated to 55° C. for 2 hrs. The mixture was concentrated under reduced pressure, the residue taken up in TFA (10 mL) and heated to reflux overnight. TFA was removed under reduced pressure and the crude product was used for next step without further purification.

MS (ES): 440 (MH⁺) for C₁₆H₂₀F₃N₃O₆S

### Intermediate 4

### Trans-N-(2-(4-aminocyclohexyl)-2-hydroxyethyl)-2-nitrobenzenesulfonamide

To a solution of trans-2,2,2-trifluoro-N-(4-(1-hydroxy-2-(2 nitrophenylsulfonamido)ethyl)cyclohexyl)acetamide (Intermediate 3, 1.58 g, 1.80 mmol) in THF (10 mL) was added K₂CO₃ (0.248 g, 1.80 mmol). The reaction was stirred at RT for 1 hour. THF was removed under reduced pressure to give the product as a colourless hard foam, 2.2 g, which was used without further purification for the next step.

MS (ES): 344 (MH⁺) for C₁₄H₂₁N₃O₅S

### Intermediate 5

### Trans-N-(2-hydroxy-2-(4-((3-oxo-3,4-dihydro-2H-pyrido[3,2-b][1,4]oxazin-6-yl)methylamino)cyclohexyl)ethyl)-2-nitrobenzenesulfonamide

To a solution of trans-N-(2-(4-aminocyclohexyl)-2-hydroxyethyl)-2-nitrobenzenesulfonamide (Intermediate 4, 2.0 g, 5.82 mmol) in MeOH (10.00 mL) was added 3-oxo-3,4-dihydro-2H-pyrido[3,2-b][1,4]oxazine-6-carbaldehyde (0.25 g, 1.40 mmol) and the mixture was heated to make a homogeneous solution. 1,2-Dichloroethane (5 mL) and molecular sieves 4 Å were added and the mixture was stirred at 67° C. for 3 hours. The mixture was cooled to room temperature, sodium triacetoxyborohydride (500 mg, 2.36 mmol) was added and the mixture was stirred at room temperature overnight. The mixture was diluted with ethyl acetate and with saturated aqueous sodium bicarbonate solution (200 ml/200 ml). The organic phase was dried over MgSO₄ and concentrated under reduced pressure to give the product as an off-white solid, (1.03 g, 35%).

MS (ES): 506 (MH⁺) for C₂₂H₂₇N₅O₇S

### Intermediate 6

### Trans-2,2,2-trifluoro-N-(4-(1-hydroxy-2-(2-nitrophenylsulfonamido)ethyl)cyclohexyl)-N-((3-oxo-3,4-dihydro-2H-pyrido[3,2-b][1,4]oxazin-6-yl)methyl)acetamide

To a solution of trans-N-(2-hydroxy-2-(4-((3-oxo-3,4-dihydro-2H-pyrido[3,2-b][1,4]oxazin-6-yl)methylamino)cyclohexyl)ethyl)-2-nitrobenzenesulfonamide (Intermediate 5, 970 mg, 1.92 mmol) in THF (10 mL) was added triethylamine (1.0 mL, 7.17 mmol) at 0° C. and 2,2,2-trifluoroacetic anhydride (280 μL, 2.01 mmol) in 5 ml of THF was added dropwise at 0° C. The mixture was stirred for 30 minutes and then allowed to warm to room temperature. More 2,2,2-trifluoroacetic anhydride (280 μL, 2.01 mmol, in 5 mL THF) was added as above and the mixture was stirred overnight at room temperature. More 2,2,2-trifluoroacetic anhydride (280 μL, 2.01 mmol, in 5 mL THF) was added and the mixture was stirred for 6 more hours at room temperature. Work up with dichloromethane/water extraction. The organic phase was dried over MgSO₄ and concentrated under reduced pressure. Chromatography was done on silica gel with 3-5% methanol in dichloromethane to give the product as a light brown solid (740 mg).

MS (ES): 602 (MH⁺) for C₂₄H₂₆N₅O₈S

### Intermediate 7

### Trans-2-(2-nitrophenylsulfonamido)-1-(4-(2,2,2-trifluoro-N-((3-oxo-3,4-dihydro-2H-pyrido[3,2-b][1,4]oxazin-6-yl)methyl)acetamido)cyclohexyl)ethyl methanesulfonate

To a mixture of trans-2,2,2-trifluoro-N-(4-(1-hydroxy-2-(2-nitrophenylsulfonamido)ethyl)cyclohexyl)-N-((3-oxo-3,4-dihydro-2H-pyrido[3,2-b][1,4]oxazin-6-yl)methyl)acetamide (Intermediate 6, 480 mg, 0.95 mmol) and triethylamine (0.2 mL, 1.44 mmol) in CH₂Cl₂ (20 mL) at RT was added methanesulfonyl chloride (25 μl, 0.32 mmol) at 0° C. and 5 mg of dimethylaminopyridine. The mixture was stirred for 2 hrs at 0° C., then concentrated to dryness and extracted with ethyl acetate. The organic layer was dried over MgSO₄ and concentrated under reduced pressure to give the crude product, which was used directly for the next step without further characterization.

### Intermediate 8

### Trans-2,2,2-trifluoro-N-4-(1-(2-nitrophenylsulfonyl-2-yl)cyclohex)-N-((3-oxo-3,4-dihydro-2H-pyrido[3,2-b][1,4]oxazin-6-yl)methyl)acetamide

To a solution of trans-2-(2-nitrophenylsulfonamido)-1-(4-(2,2,2-trifluoro-N-((3-oxo-3,4-dihydro-2H-pyrido[3,2-b][1,4]oxazin-6-yl)methyl)acetamido)cyclohexyl)ethyl methanesulfonate (Intermediate 7, 645 mg, 0.95 mmol) in THF (15 mL) at room temperature was added K₂CO₃ (500 mg, 3.62 mmol) and water 1.0 mL and the mixture was heated to 60° C. for two days, then for 12 hrs at 75° C. THF was removed under reduced pressure and the residue was taken up with dichloromethane, washed with brine, then dried over magnesium sulfate and concentrated under reduced pressure. Chromatography was done on silica gel with 0 go 3% methanol in dichloromethane. The sample was further purified by reverse phase HPLC with acetonitrile/water to give the product as a colourless solid, 38 mg.

MS (ES): 584 (MH⁺) for C₂₄H₂₄N₅O₇S

### Intermediate 9

### Trans-2,2,2-trifluoro-N-(4-(2-(7-methoxy-2-oxo-1,5-naphthyridin-1(2H)-yl)-1-(2-nitrophenylsulfonamido)ethyl)cyclohexyl)-N-((3-oxo-3,4-dihydro-2H-pyrido[3,2-b][1,4]oxazin-6-yl)methyl)acetamide

To a solution of 7-methoxy-1,5-naphthyridin-2(1H)-one (prepared according to the procedure described in WO07/013,8974; 57 mg, 0.32 mmol) in DMF (3 mL) was added NaH (13 mg, 0.33 mmol) at room temperature under nitrogen. The mixture was stirred for 15 minutes, then a solution of trans-2,2,2-trifluoro-N-(4-(1-(2-nitrophenylsulfonyl)aziridin-2-yl)cyclohexyl)-N-((3-oxo-3,4-dihydro-2H-pyrido[3,2-b][1,4]oxazin-6-yl)methyl)acetamide (Intermediate 8, 38 mg, 0.07 mmol) in DMF (3 mL) was added dropwise and the mixture was stirred for 2 hours at room temperature. The mixture of the crude product was used directly for the next step.

MS (ES): 760 (MH⁺) for C₃₃H₃₂F₃N₇O₉S

### Intermediate 10

### Trans-N-(4-(1-amino-2-(7-methoxy-2-oxo-1,5-naphthyridin-1(2H)-yl)ethyl)cyclohexyl)-2,2,2-trifluoro-N-((3-oxo-3,4-dihydro-2H-pyrido[3,2-b][1,4]oxazin-6-yl)methyl)acetamide

To the crude solution of trans-2,2,2-trifluoro-N-(4-(2-(7-methoxy-2-oxo-1,5-naphthyridin-1(2H)-yl)-1-(2-nitrophenylsulfonamido)ethyl)cyclohexyl)-N4(3-oxo-3,4-dihydro-2H-pyrido[3,2-b][1,4]oxazin-6-yl)methyl)acetamide (Intermediate 9, ˜53 mg, 0.07 mmol) in DMF (5 mL) was added benzenethiol (25 μl, 0.07 mmol) and K₂CO₃ (10 mg) at room temperature under nitrogen. The reaction mixture was stirred for 30 minutes at room temperature. DMF was removed under reduced pressure, providing the title product as a racemic mixture. The crude product was used without further purification for the next step.

MS (ES): 575 (MH⁺) for C₂₇H₂₉F₃N₆O₅

### Intermediate 11

### Trans-tert-Butyl 4-(1(2-nitrophenylsulfonyl)aziridin-2-yl)-cyclohexylcarbamate

To a solution of copper (II) triflate (643, 1.78 mmol) in dry acetonitrile (2 mL) was added a solution of tert-butyl 4-vinylcyclohexylcarbamate (4.0 g, 17.75 mmol) in dry acetonitrile (4 mL, heated till dissolved). The mixture was warmed gently to enhance solubility, then cooled to room temperature and [N-(o-nitrophenylsulfonyl)imino]phenyliodinane (prepared according to the procedure described in Tetrahedron Letters, Vol. 38, No. 39, pp. 6897-6900, 1997; 7.2 g, 17.75 mmol) was added. The reaction mixture became exothermic after several minutes. The mixture was stirred at room temperature under nitrogen for three hours, then concentrated under reduced pressure. The crude product was taken up in dichloromethane and the solids were removed by filtration. The filtrate was chromatographed on silica gel eluting with 10-50% acetone in hexanes to give 2.0 g (27%) of the title product as a racemic mixture in the form of an off-white solid.

MS (ES): 426 (MH⁺) for C₁₉H₂₇N₃O₆S

¹H NMR (DMSO-d₆) δ: ppm 0.98-1.12 (m, 5H); 1.36 (m, 10H); 1.45-1.53 (m, 1H); 1.60-1.65 (m, 1H); 1.66-1.79 (m, 2H); 2.63-2.69 (m, 1H); 2.68-2.75 (m, 1H); 3.09 (brs, 1H); 6.64 (d, 1H); 7.88-7.95 (m, 1H); 7.96-8.02 (m, 1H); 8.03-8.07 (m, 1H); 8.15 (dd, 1H).

The R and S enantiomers of Intermediate 11 were separated by chiral HPLC (Chiralcel OJ column, 20×250 mm, 10μ, 1:1 isopropanol:hexanes, 10 mL/min) to give Intermediate 11(a) as first eluting enantiomer and Intermediate 11(b) as second eluting enantiomer.

### Intermediate 11(a), First Eluting Compound

### Trans-tert-Butyl 4-(1-(2-nitrophenylsulfonyl)aziridin-2-yl)--cyclohexylcarbamate, (+) enantiomer

[α]_{D}=+39.7 (c=1 in methanol).

### Intermediate 11(b), Second Eluting Compound

### Trans-tert-Butyl 4-(1-(2-nitrophenylsulfonyl)aziridin-2-yl)-cyclohexylcarbamate, (−) enantiomer

The second eluting enantiomer was collected and concentrated under reduced pressure to give the title product as a colorless hard foam. [α]_{D}=−62.5 (c=0.2 in CHCl₃).

### Intermediate 12

### Trans-tert-Butyl 4-(2-(7-methoxy-2-oxo-1,5-naphthyridin-1(2H)-yl)-1-(2-nitrophenylsulfonamido)ethyl)-cyclohexylcarbamate

7-Methoxy-1,5-naphthyridin-2(1H)-one (prepared according to the procedure described in PCT Pub. No. WO07/013,8974; 444 mg, 2.52 mmol), sodium hydride (60% dispersion in mineral oil) (101 mg, 2.52 mmol), and trans-tert-butyl 4-(1-(2-nitrophenylsulfonyl)aziridin-2-yl)-cyclohexylcarbamate (Intermediate 11, 715 mg, 1.68 mmol) were reacted as described for Intermediate 15 to give 740 mg (73%) of the title product as a racemic mixture in the form of an off-white solid.

MS (ES): 602 (MH⁺) for C₂₈H₃₅N₅O₈S

¹H NMR (DMSO-d₆) δ: ppm 1.01-1.20 (m, 4H); 1.38 (s, 9H); 1.47-1.59 (m, 1H); 1.73-1.94 (m, 4H); 3.14 (brs, 1H); 3.66-3.78 (m, 1H); 3.98 (s, 3H); 4.12 (brs, 1H); 4.27-4.39 (m, 1H); 6.48-6.58 (m, 1H); 6.65-6.75 (m, 1H); 7.39-7.45 (m, 1H); 7.47-7.69 (m, 5H); 7.76-7.89 (m, 1H); 8.11 (s, 1H).

### Intermediate 12(a)

### Trans-tert-butyl 4-(2-(7-methoxy-2-oxo-1,5-naphthyridin-1(2H)-yl)-1-(2-nitrophenylsulfonamido)ethyl)-cyclohexylcarbamate, (−) enantiomer

The title product was chirally synthesized as a single enantiomer from trans-tert-butyl 4-(1-(2-nitrophenylsulfonyl)aziridin-2-yl)-cyclohexylcarbamate, (−) enantiomer (Intermediate 11(b)) using a procedure similar to the one described for the synthesis of Intermediate 12.

[α]_{D}=−15.5 (c=0.2 in CHCl₃).

### Intermediate 13

### Trans-N-(1-(4-aminocyclohexyl)-2-(7-methoxy-2-oxo-1,5-naphthyridin-1(2H)-yl)ethyl)-2-nitrobenzenesulfonamide, trifluoro acetic acid salt

Trans-tert-butyl 4-(2-(7-methoxy-2-oxo-1,5-naphthyridin-1(2H)-yl)-1-(2-nitrophenylsulfonamido)ethyl)-cyclohexylcarbamate (Intermediate 12, 655 mg, 1.09 mmol) was reacted with trifluoroacetic acid in dichloromethane as described for Intermediate 16 to give 670 mg (quant.) of the title product as a racemic mixture that was used without further purification.

MS (ES): 502 (MH⁺) for C₂₃H₂₇N₅O₆S

### Intermediate 13(a)

### Trans-N-(1-(4-aminocyclohexyl)-2-(7-methoxy-2-oxo-1,5-naphthyridin-1(2H)-yl)ethyl)-2-nitrobenzenesulfonamide, enantiomer A, trifluoro acetic acid salt

The title product was chirally synthesized as a single enantiomer from trans-tert-butyl 4-(2-(7-methoxy-2-oxo-1,5-naphthyridin-1(2H)-yl)-1-(2-nitrophenylsulfonamido)ethyl)-cyclohexylcarbamate, enantiomer A (Intermediate 12(a)) using a procedure similar to the one described for the synthesis of Intermediate 13.

### Intermediate 14

### Trans-N-(2-(7-methoxy-2-oxo-1,5-naphthyridin-1(2H)-yl)-1-(4-((3-oxo-3,4-dihydro-2H-pyrido[3,2-b][1,4]oxazin-6-yl)methylamino)-cyclohexyl)ethyl)-2-nitrobenzenesulfonamide

A mixture of trans-N-(1-(4-aminocyclohexyl)-2-(7-methoxy-2-oxo-1,5-naphthyridin-1(2H)-yl)ethyl)-2-nitrobenzenesulfonamide, trifluoro acetic acid salt (Intermediate 13, 795 mg, 1.09 mmol), 3-oxo-3,4-dihydro-2H-pyrido[3,2-b][1,4]oxazine-6-carbaldehyde (prepared according to the procedure described in PCT Pub. No. WO04/058144; 194 mg, 1.09 mmol) and ethyl(diisopropyl)amine (758 μL, 4.36 mmol) in methanol/chloroform (1:1, 20 mL) over 3 Å molecular sieves was heated to 70° C. for two hours under nitrogen. The reaction mixture was cooled to room temperature and treated with sodium triacetoxy borohydride (693 mg, 3.27 mmol). After stirring for 2 hours, the reaction was diluted with 15% methanol in dichloromethane and filtered through diatomaceous earth (Celite® brand). The filtrate was washed with saturated sodium bicarbonate solution. The aqueous phase was re-extracted once with 15% methanol/dichloromethane. The combined organic phases were dried over sodium sulfate and concentrated under reduced pressure. Chromatography on silica gel eluting with 2-10% methanol in dichloromethane containing 0.25% ammonium hydroxide gave 555 mg (77%) of the title product as a racemic mixture in the form of an off-white solid.

MS (ES): 664 (MH⁺) for C₃₁H₃₃N₇O₈S

¹H NMR (DMSO-d₆) δ: ppm 0.98-1.13 (m, 4H); 1.57 (brs, 1H); 1.86-2.01 (m, 4H); 2.30 (brs, 1H); 3.68-3.77 (m, 2H); 3.99 (s, 3H); 4.12 (brs, 1H); 4.26-4.37 (m, 1H); 4.61 (s, 2H); 6.52 (d, 1H); 7.01 (d, 1H); 7.30 (d, 1H); 7.40-7.64 (m, 6H); 7.77 (brs, 1H); 8.09 (d, 1H); 11.15 (brs, 1H).

### Intermediate 14(a)

### Trans-N-(2-(7-methoxy-2-oxo-1,5-naphthyridin-1(2H)-yl)-1-(4-((3-oxo-3,4-dihydro-2H-pyrido[3,2-b][1,4]oxazin-6-yl)methylamino)-cyclohexyl)ethyl)-2-nitrobenzenesulfonamide, (+) enantiomer

The title product was chirally synthesized as a single enantiomer from trans-N-(1-(4-aminocyclohexyl)-2-(7-methoxy-2-oxo-1,5-naphthyridin-1(2H)-yl)ethyl)-2-nitrobenzenesulfonamide, enantiomer A, trifluoro acetic acid salt (Intermediate 13(a)) using a procedure similar to the one described for the synthesis of Intermediate 14.

[α]_{D}=+13.5 (c=0.2 in CHCl₃).

### Intermediate 15

### Trans-tert-butyl 4-(2-(7-methoxy-2-oxoquinoxalin-1(2H)-yl)-1-(2-nitrophenylsulfonamido)ethyl)-cyclohexylcarbamate

To a solution of 7-methoxyquinoxalin-2(1H)-one (prepared according to the procedure described in PCT Pub. No. WO08/071,961; 397 mg, 2.26 mmol) in dry DMF (10 mL) was added sodium hydride (60% dispersion in mineral oil; 113 mg, 2.82 mmol) with stirring under nitrogen. After 30 minutes a solution of trans-tert-butyl 4-(1-(2-nitrophenylsulfonyl)aziridin-2-yl)-cyclohexylcarbamate (Intermediate 11, 800 mg, 1.88 mmol) in dry DMF (3 mL) was added and the mixture was stirred at room temperature for 15 hours. The mixture was quenched with potassium phosphate buffer pH 7 (1M, 2 mL). The reaction mixture was partitioned between ethyl acetate and water, the layers were separated and the aqueous phase was back-extracted once with ethyl acetate. The combined organic phases were washed with water (3×), followed by brine (1×), dried over sodium sulfate and concentrated under reduced pressure. The residue was triturated with hot toluene and the solid was collected by filtration to give 570 mg of the title product. The filtrate was concentrated in vacuo and the resulting crude material subjected to chromatography on silica gel eluting with 10-50% acetone in hexanes to give an additional 153 mg (64% total yield) of product as a racemic mixture in the form of an off white solid.

MS (ES): 602 (MH⁺) for C₂₈H₃₅N₅O₈S

¹H NMR (DMSO-d₆) δ: ppm 0.98-1.18 (m, 4H); 1.37 (s, 9H); 1.45-1.58 (m, 1H); 1.72-1.92 (m, 4H); 3.10 (brs, 1H); 3.73 (brs, 1H); 3.92 (s, 3H); 4.13-4.31 (m, 2H); 6.61-6.71 (m, 1H); 6.86 (dd, 1H); 7.04 (s, 1H); 7.46-7.68 (m, 5H); 7.86-7.97 (m, 2H).

### Intermediate 15(a)

### Trans-tert-butyl 4-(2-(7-methoxy-2-oxoquinoxalin-1(2H)-yl)-1-(2-nitrophenylsulfonamido)ethyl)-cyclohexylcarbamate, (+) enantiomer

The title product was chirally synthesized as a single enantiomer from trans-tert-butyl 4-(1-(2-nitrophenylsulfonyl)aziridin-2-yl)-cyclohexylcarbamate, (−) enantiomer (Intermediate 11(b)) using a procedure similar to the one described for the synthesis of Intermediate 15.

[α]_{D}=+6.0 (c=0.1 in methanol).

### Intermediate 16

### Trans-N-(1-(4-aminocyclohexyl)-2-(7-methoxy-2-oxoquinoxalin-1(2H)-yl)ethyl)-2-nitrobenzenesulfonamide, trifluoroacetic acid salt

A solution of trans-tert-butyl 4-(2-(7-methoxy-2-oxoquinoxalin-1(2H)-yl)-1-(2-nitrophenylsulfonamido)ethyl)-cyclohexylcarbamate (Intermediate 15, 570 mg, 0.95 mmol) in dichloromethane (10 mL) was treated with trifluoroacetic acid (3 mL). After 1 hour the reaction was concentrated under reduced pressure. The crude product was co-evaporated 2× with dichloromethane giving 580 mg (quant.) of the title product as a racemic mixture that was used in the next step without further purification.

MS (ES): 502 (MH⁺) for C₂₃H₂₇N₅O₆S

### Intermediate 16(a)

### Trans-N-(1-(aminocyclohexyl)-2-(7-methoxy-2-oxoquinoxalin-1(2H)-yl)ethyl)-2-nitrobenzenesulfonamide, enantiomer A, trifluoroacetic acid salt

The title product was chirally synthesized as a single enantiomer from trans-tert-butyl 4-(2-(7-methoxy-2-oxoquinoxalin-1(2H)-yl)-1-(2-nitrophenylsulfonamido)ethyl)-cyclohexylcarbamate, enantiomer A (Intermediate 15(a)) using a procedure similar to the one described for the synthesis of Intermediate 16.

### Intermediate 17

### Trans-N-(2-(7-methoxy-2-oxoquinoxalin-1(2H)-yl)-1-(4-((3-oxo-3,4-dihydro-2H-pyrido[3,2-b][1,4]oxazin-6-yl)methylamino)-cyclohexyl)ethyl)-2-nitrobenzenesulfonamide

A solution of trans-N-(1-(4-aminocyclohexyl)-2-(7-methoxy-2-oxoquinoxalin-1(2H)-yl)ethyl)-2-nitrobenzenesulfonamide, trifluoroacetic acid salt (Intermediate 16, 583 mg, 0.95 mmol), 3-oxo-3,4-dihydro-2H-pyrido[3,2-b][1,4]oxazine-6-carbaldehyde (prepared according to the procedure described in PCT Pub. No. WO04/058144; 169 mg, 0.95 mmol), ethyl(diisopropyl)amine (659 mL, 3.79 mmol) and sodium triacetoxy borohydride (602 mg, 2.84 mmol) in DMF (20 mL) was heated at 40° C. under nitrogen over 3 Å molecular sieves for 15 hours. The reaction was cooled to room temperature, diluted with 1:1 methanol/dichloromethane, filtered through diatomaceous earth (Celite® brand) and concentrated under reduced pressure. The residue was partitioned between 15% methanol in dichloromethane and saturated sodium bicarbonate solution. The aqueous phase was back-extracted once with 15% methanol in dichloromethane. The combined organic phases were dried over sodium sulfate and concentrated under reduced pressure to give 790 mg of the crude product as a racemic mixture which was used in the next step without further purification.

MS (ES): 664 (MH⁺) for C₃₁H₃₃N₇O₈S

¹H NMR (DMSO-d₆) δ: ppm 0.94-1.12 (m, 4H); 1.50-1.61 (m, 1H); 1.83-1.98 (m, 4H); 2.25-2.37 (m, 1H); 3.67-3.76 (m, 2H); 3.92 (s, 3H); 4.12-4.30 (m, 2H); 4.61 (s, 2H); 6.81-6.88 (m, 1H); 6.98-7.06 (m, 2H); 7.27-7.32 (m, 1H); 7.45-7.64 (m, 6H); 7.89 (s, 1H); 11.19 (brs, 1H).

### Intermediate 17(a)

### Trans-N-(2-(7-methoxy-2-oxoquinoxalin-1(2H)-yl)-1-(4-((3-oxo-3,4-dihydro-2H-pyrido[3,2-b][1,4]oxazin-6-yl)methylamino)-cyclohexyl)ethyl)-2-nitrobenzenesulfonamide, (+) enantiomer

The title product was chirally synthesized as a single enantiomer from trans-N-(1-(4-aminocyclohexyl)-2-(7-methoxy-2-oxoquinoxalin-1(2H)-yl)ethyl)-2-nitrobenzenesulfonamide, enantiomer A, trifluoroacetic acid salt (Intermediate 16(a)) using a procedure similar to the one described for the synthesis of Intermediate 17.

[α]_{D}=+18 (c=0.1 in methanol)

### Intermediate 18

### Trans-N-(2-(7-methoxy-2-oxoquinoxalin-1(2H)-yl)-1-(4-((7-oxo-7,8-dihydro-6H-pyrimido[5,4-b][1,4]oxazin-2-yl)methylamino)-cyclohexyl)ethyl)-2-nitrobenzenesulfonamide

trans-N-(1-(4-aminocyclohexyl)-2-(7-methoxy-2-oxoquinoxalin-1(2H)-yl)ethyl)-2-nitrobenzenesulfonamide, trifluoroacetic acid salt (Intermediate 16, 815 mg, 1.32 mmol), 7-oxo-7,8-dihydro-6H-pyrimido[5,4-b][1,4]oxazine-2-carbaldehyde (prepared according to the procedure described in PCT Pub. No. WO08/009,700; 237 mg, 1.32 mmol), and sodium triacetoxy borohydride (842 mg, 3.97 mmol) were reacted as described for Intermediate 17 to give 860 mg of crude product as a racemic mixture, which was used in the next step without further purification.

MS (ES): 665 (MH⁺) for C₃₀H₃₂N₈O₈S

¹H NMR (DMSO-d₆) δ: ppm 0.93-1.18 (m, 4H); 1.55 (brs, 1H); 1.80-1.98 (m, 4H); 2.35 (brs, 1H); 3.69-3.80 (m, 2H); 3.92 (s, 3H); 4.10-4.31 (m, 2H); 4.72 (s, 2H); 6.84 (dd, 1H); 7.04 (s, 1H); 7.43-7.66 (m, 5H); 7.89 (s, 1H); 7.96 (s, 1H); 8.23 (s, 1H).

### Intermediate 18(a)

### N-(2-(7-methoxy-2-oxoquinoxalin-1(2H)-yl)-1-(4-((7-oxo-7,8-dihydro-6H-pyrimido[5,4-b][1,4]oxazin-2-yl)methylamino)-cyclohexyl)ethyl)-2-nitrobenzenesulfonamide, enantiomer A

The title product was chirally synthesized as a single enantiomer from trans-N-(1-(4-aminocyclohexyl)-2-(7-methoxy-2-oxoquinoxalin-1(2H)-yl)ethyl)-2-nitrobenzenesulfonamide, trifluoroacetic acid salt, enantiomer A (Intermediate 16(a)) using a procedure similar to the one described for the synthesis of Intermediate 17.

### Intermediate 19

### Trans-tert-butyl-4-(2-(7-cyano-2-oxoquinolin-1(2H)-yl)-1-(2-nitrophenylsulfonamido)ethyl)-cyclohexylcarbamate

To a solution of 2-oxo-1,2-dihydroquinoline-7-carbonitrile (prepared according to the procedure described in PCT Pub. No. WO08/007,196; 0.624 g, 3.67 mmol) in DMF (35 mL) at room temperature was added sodium hydride (60% in mineral oil, 0.147 g, 3.67 mmol). Trans-tert-Butyl 4-(1-(2-nitrophenylsulfonyl)aziridin-2-yl)-cyclohexylcarbamate (Intermediate 11, 1.3 g, 3.06 mmol) was added and the mixture was stirred at room temperature overnight. The reaction mixture was diluted with water, extracted twice with dichloromethane, dried over magnesium sulfate and concentrated under reduced pressure. Silica gel chromatography (0%-80% ethyl acetate/hexanes) afforded the title product as a racemic mixture, 1.287 g (70%).

MS (ES): 594 (M−H⁻) for C₂₉H₃₃N₅O₇S

¹H NMR (DMSO-d₆) δ: 1.09 (m, 4H); 1.36 (s, 9H); 1.54 (m, 1H); 1.81 (m, 4H); 3.12 (m, 1H); 3.70 (m, 1H); 4.13 (m, 1H); 4.28 (m, 1H); 6.62 (d, 1H); 6.70 (m, 1H); 7.51 (m, 6H); 7.70 (d, 1H); 7.85 (m, 1H); 8.19 (s, 1H).

### Intermediate 20

### Trans-N-(1-(4-aminocyclohexyl)-2-(7-cyano-2-oxoquinolin-1(2H)-yl)ethyl)-2-nitrobenzenesulfonamide, trifluoroacetic acid salt

Trans-tert-butyl-4-(2-(7-cyano-2-oxoquinolin-1(2H)-yl)-1-(2-nitrophenylsulfonamido)ethyl)-cyclohexylcarbamate (Intermediate 19, 1.287 g, 2.16 mmol) was reacted with trifluoroacetic acid (1.998 mL, 25.93 mmol) in dichloromethane as described for Intermediate 16 to give the title product as a racemic mixture in the form of an off-white solid, 1.42 g.

MS (ES): 496 (MH⁺) for C₂₄H₂₅N₅O₅S

### Intermediate 21

### Trans-N-(2-(7-cyano-2-oxoquinolin-1(2H)-yl)-1-(4-((3-oxo-3,4-dihydro-2H-pyrido[3,2-b][1,4]oxazin-6-yl)methylamino)-cyclohexyl)ethyl)-2-nitrobenzenesulfonamide

A mixture of trans-N-(1-(4-aminocyclohexyl)-2-(7-cyano-2-oxoquinolin-1(2H)-yl)ethyl)-2-nitrobenzenesulfonamide (Intermediate 20, 0.535 g, 1.08 mmol) and 3-oxo-3,4-dihydro-2H-pyrido[3,2-b][1,4]oxazine-6-carbaldehyde (prepared according to the procedure described in PCT Pub. No. WO04/058144; 0.288 g, 1.62 mmol) in chloroform (10 mL) and methanol (20 mL) was heated over 3 Å molecular sieves at 70° C. for 4 hours. The reaction mixture was then cooled to 0° C. and sodium triacetoxyborohydride (0.458 g, 2.16 mmol) was added. After stirring at room temperature for 2 hours, the reaction was quenched with saturated sodium bicarbonate and filtered. The filtrate was diluted with dichloromethane, the phase was dried over magnesium sulfate and concentrated under reduced pressure. Silica gel chromatography (0%-10% methanol in dichloromethane) afforded the title product as a racemic mixture in the form of a colourless solid, 0.563 g (79%).

MS (ES): 658 (MH⁺) for C₃₂H₃₁N₇O₇S

¹H NMR (DMSO-d₆) δ: 1.25 (m, 6H); 1.65 (m, 1H); 1.99 (m, 2H); 2.17 (m, 2H); 3.04 (m, 1H); 3.76 (m, 1H); 4.12 (m, 2H); 4.29 (m, 1H); 4.69 (s, 2H); 6.60 (d, 1H); 7.11 (d, 1H); 7.36 (m, 1H); 7.44 (m, 3H); 7.54 (m, 3H); 7.68 (d, 1H); 7.86 (m, 1H); 8.19 (s, 1H); 11.34 (s, 1H).

### Intermediate 22

### Trans-N-(2-(7-fluoro-2-oxoquinoxalin-1(2H)-yl)-1-(4-((3-oxo-3,4-dihydro-2H-pyrido[3,2-b][1,4]oxazin-6-yl)methylamino)cyclohexyl)ethyl)-2-nitrobenzenesulfonamide, enantiomer A

Trans-N-(1-(4-aminocyclohexyl)-2-(7-fluoro-2-oxoquinoxalin-1(2H)-yl)ethyl)-2-nitrobenzenesulfonamide, enantiomer A, trifluoracetic acid salt (Intermediate 23, 142 mg, 0.24 mmol) was dissolved in DMF (3 mL). Diisopropylethylamine (0.123 mL, 0.71 mmol) and freshly activated MS3A (perled) was added under nitrogen, followed by addition of 3-oxo-3,4-dihydro-2H-pyrido[3,2-b][1,4]oxazine-6-carbaldehyde (41.9 mg, 0.24 mmol) (prepared according to the procedure described in PCT Pub. No. WO04/058144) and the mixture was heated to 40° C. for 15 minutes. Sodium triacetoxyborohydride (125 mg, 0.59 mmol) was added and the mixture was heated at 40° C. over night. The mixture was filtred through a 0.45 um membrane, the filter cake was washed with methanol/dichloromethane (1:1) and the combined filtrate and wash were concentrated under reduced pressure to dryness. DMF was removed by codistillation with toluene. The residue was taken up in dichloromethane (˜5 mL) and loaded onto a silica gel column with dead space, eluting with CH₂Cl₂/MeOH 15:1, containing 0.25% ammonium hydroxide. Fractions containing product were pooled and concentrated under reduced pressure to give 123 mg of the title product as a single enantiomer in the form of a colorless hard foam.

MS (ES): 652 (MH⁺) for C₃₀H₃₀FN₇O₇S

¹H NMR (DMSO-d₆) δ: 11.15 (brs, 1H); 8.04 (s, 1H); 7.89 (brs, 1H); 7.66-7.49 (m, 6H); 7.30 (d, 1H); 7.09 (m, 1H); 7.02 (d, 1H); 4.61 (s, 2H); 4.24 (m, 1H); 4.08 (m, 1H); 3.71 (m, 3H); 2.31 (m, 1H); 1.99-1.75 (m, 4H); 1.57 (m, 1H); 1.16-0.88 (m, 4H).

### Intermediate 23

### Trans-N-(1-(4-aminocyclohexyl)-2-(7-fluoro-2-oxoquinoxalin-1(2H)-yl)ethyl)-2-nitrobenzenesulfonamide, enantiomer A, trifluoroacetic acid salt

A solution of trans-tert-butyl 4-(2-(7-fluoro-2-oxoquinoxalin-1(2H)-yl)-1-(2-nitrophenylsulfonamido)ethyl)cyclohexylcarbamate, enantiomer A (Intermediate 24, 295 mg, 0.50 mmol) in dichloromethane (6 ml) at 0° C. was treated with trifluoroacetic acid (3 mL). The cooling bath was removed and the mixture was stirred at room temperature for 2 hours. The mixture was concentrated under reduced pressure, then codistilled with dichloromethane (twice) and then with methanol (twice) to give the title product, 233 mg, as a single enantiomer in the form of a colourless oil.

MS (ES): 490 (MH⁺) for C₂₂H₂₄FN₅O₅S

### Intermediate 24

### Trans-tert-butyl 4-(2-(7-fluoro-2-oxoquinoxalin-1(2H)-yl)-1-(2-nitrophenylsulfonamido)ethyl)cyclohexylcarbamate, enantiomer A

To a mixture of 7-fluoroquinoxalin-2(1H)-one (prepared according to the procedure described in PCT Pub. No. WO08/071,961; 171 mg, 1.04 mmol) in DMF (5 mL) was added sodium hydride (49.9 mg, 1.25 mmol) and the mixture was stirred at room temperature for 15 minutes under nitrogen. A solution of trans-tert-butyl 4-(1-(2-nitrophenylsulfonyl)aziridin-2-yl)cyclohexylcarbamate, (−) enantiomer (Intermediate 11(b), 442 mg, 1.04 mmol) in DMF (5 mL) was added and the mixture was stirred at room temperature over night. The mixture was quenched with phosphate buffer pH 7 (30 mL, 1M) and extracted with ethyl acetate (100 mL). The organic phase was washed with water twice (2×100 mL) and dried over sodium sulfate, then concentrated under reduced pressure. Chromatography was done on silica gel with hexanes/acetone 2:1 to give 300 mg of the title product as a single enantiomer in the form of colorless solid.

MS (ES): 612 (MNa⁺) for C₂₇H₃₂FN₅O₇S

¹H NMR (DMSO-d₆) δ: 8.05 (s, 1H); 7.92 (d, 1H); 7.68-7.52 (m, 6H); 7.11 (ddd, 1H); 6.66 (d, 1H); 4.23 (dd, 1H); 4.09 (dd, 1H); 3.69 (m, 1H); 3.12 (m, 1H); 1.86-1.71 (m, 4H); 1.51 (m, 1H); 1.38 (s, 9H); 1.22-0.94 (m, 4H).

### Intermediate 25

### Trans-N-(2-(7-fluoro-2-oxoquinoxalin-1(2H)-yl)-1-(4-((7-oxo-7,8-dihydro-6H-pyrimido[5,4-b][1,4]oxazin-2-yl)methylamino)cyclohexyl)ethyl)-2-nitrobenzenesulfonamide, enantiomer A

Trans-N-(1-(4-aminocyclohexyl)-2-(7-fluoro-2-oxoquinoxalin-1(2H)-yl)ethyl)-2-nitrobenzenesulfonamide, enantiomer A, trifluoroacetic acid salt (Intermediate 23, 143 mg, 0.24 mmol) was reacted with DIEA (0.123 mL, 0.71 mmol), 7-oxo-7,8-dihydro-6H-pyrimido[5,4-b][1,4]oxazine-2-carbaldehyde (prepared according to the procedure described in PCT Pub. No. WO08/009,700) (42.4 mg, 0.24 mmol) and sodium triacetoxyborohydride (125 mg, 0.59 mmol) as described for Intermediate 22 to give 106 mg of the title product as a single enantiomer in the form of a colorless hard foam.

MS (ES): 653 (MH⁺) for C₂₉H₂₉FN₈O₇S

¹H NMR (DMSO-d₆) δ: 8.25 (s, 1H); 8.04 (s, 1H); 7.87 (brs, 1H); 7.66-7.49 (m, 6H); 7.08 (ddd, 1H); 4.73 (s, 2H); 4.23 (dd, 1H); 4.08 (m, 1H); 3.76 (s, 2H); 3.69 (m, 1H); 2.35 (m, 1H); 1.99-1.77 (m, 4H); 1.58 (m, 1H); 1.18-0.91 (m, 4H).

### Intermediate 26

### Trans-N-(2-(7-cyano-2-oxoquinolin-1(2H)-yl)-1-(4-((3-oxo-3,4-dihydro-2H-pyrido[3,2-b][1,4]thiazin-6-yl)methylamino)-cyclohexyl)ethyl)-2-nitrobenzenesulfonamide

Trans-N-(1-(4-aminocyclohexyl)-2-(7-cyano-2-oxoquinolin-1(2H)-yl)ethyl)-2-nitrobenzenesulfonamide, trifluoroacetic acid salt (Intermediate 20, 0.535 g, 1.08 mmol) and 3-oxo-3,4-dihydro-2H-pyrido[3,2-b][1,4]thiazine-6-carbaldehyde (prepared according to the procedure described in PCT Pub. No. WO 2004/058144, 0.315 g, 1.62 mmol) were reacted with sodium triacetoxyborohydride (0.458 g, 2.16 mmol) as described for Intermediate 21 to give the title product as a racemic mixture in the form of an orange solid, 0.439 g, 60%.

MS (ES): 674 (MH⁺) for C₃₂H₃₁N₇O₆S₂

¹H NMR (DMSO-d₆) δ: 1.04 (m, 4H); 1.60 (m, 1H); 1.88 (m, 4H); 2.34 (m, 1H); 3.52 (s, 2H); 3.75 (m, 3H); 4.12 (m, 1H); 4.29 (m, 1H); 6.62 (d, 1H); 7.09 (d, 1H); 7.42 (m, 3H); 7.55 (m, 4H); 7.71 (m, 2H); 7.83 (m, 1H); 8.20 (s, 1H); 10.88 (s, 1H).

### Intermediate 27

### Trans-N-(2-(7-methoxy-2-oxo-1,5-naphthyridin-1(2H)-yl)-1-(4-((7-oxo-7,8-dihydro-6H-pyrimido[5,4-b][1,4]oxazin-2-yl)methylamino)cyclohexyl)ethyl)-2-nitrobenzenesulfonamide

A solution of trans-N-(1-(4-aminocyclohexyl)-2-(7-methoxy-2-oxo-1,5-naphthyridin-1(2H)-yl)ethyl)-2-nitrobenzenesulfonamide trifluoroacetate (Intermediate 13, 570 mg, 0.93 mmol), 7-oxo-7,8-dihydro-6H-pyrimido[5,4-b][1,4]oxazine-2-carbaldehyde (prepared according to the procedure described in PCT Pub. No. WO08/009,700, 166 mg, 0.93 mmol), and ethyl(diisopropyl)amine (644 μL, 3.70 mmol) in DMF (10 mL) was heated to 45° C. under nitrogen over 3 Å molecular sieves for 15 minutes. Sodium triacetoxy borohydride (589 mg, 2.78 mmol) was added and the reaction was stirred at 45° C. for 16 hours. The reaction mixture was cooled to room temperature, diluted with 1:1 methanol/dichloromethane and filtered through diatomaceous earth (Celite® brand). The filtrate was concentrated under reduced pressure. The crude product was partitioned between 15% methanol in dichloromethane and saturated sodium bicarbonate solution. The layers were separated and the aqueous phase was re-extracted once with 15% methanol in dichloromethane. The combined organic layers were dried over sodium sulfate and concentrated under reduced pressure. Chromatography was done on silica gel with 5-10% methanol in dichloromethane containing 0.25% ammonium hydroxide to give 500 mg (81%) of the title product as a racemic mixture in the form of a solid.

MS (ES): 665 (MH⁺) for C₃₀H₃₂N₈O₈S

¹H NMR (DMSO-d₆) δ ppm 0.95-1.18 (m, 4H); 1.58 (brs, 1H); 1.85-2.02 (m, 4H); 2.37 (brs, 1H); 3.71 (brs, 1H); 3.77 (s, 2H); 3.97 (s, 3H); 4.12 (brs, 1H); 4.25-4.37 (m, 1H); 4.73 (s, 2H); 6.52 (d, 1H); 7.38-7.65 (m, 7H); 8.09 (m, 1H); 8.25 (s, 1H).

### Intermediate 28

### Trans-N-(2-(7-fluoro-2-oxo-1,5-naphthyridin-1(2H)-yl)-1-(4-((3-oxo-3,4-dihydro-2H-pyrido[3,2-b][1,4]oxazin-6-yl)methylamino)cyclohexyl)ethyl)-2-nitrobenzenesulfonamide, enantiomer A

Trans-N-(1-(4-aminocyclohexyl)-2-(7-fluoro-2-oxo-1,5-naphthyridin-1(2H)-yl)ethyl)-2-nitrobenzenesulfonamide, trifluoroacetic acid salt, enantiomer A (Intermediate 29, 140 mg, 0.29 mmol) was dissolved in DMF (3 mL), diisopropylethyl amine (148 mg, 1.14 mmol) was added, followed by addition of 3-oxo-3,4-dihydro-2H-pyrido[3,2-b][1,4]oxazine-6-carbaldehyde (prepared according to the procedure described in PCT Pub. No. WO04/058144; 51 mg, 0.29 mmol). The mixture was heated to 40° C. for 15 minutes over freshly activated molecular sieves 3 A. Sodium triacetoxy-borohydride (182 mg, 0.82 mmol) was added and the mixture was stirred at 40° C. overnight. The mixture was cooled to room temperature, diluted with 1:1 methanol/dichloromethane and filtered through diatomaceous earth (Celite® brand). The filtrate was concentrated under reduced pressure. The residue was dissolved in 15% methanol in dichloromethane and washed with saturated sodium bicarbonate. The layers were separated and the aqueous phase was re-extracated once. The combined organic phases were dried over sodium sulfate and concentrated under reduced pressure. Chromatography was done on silica gel with 0-2% methanol in dichloromethane, containing 0.25% ammonium hydroxide to give 65 mg of the title product as a single enantiomer in the form of an off-white solid.

MS (ES): 652 (MH⁺) for C₃₀H₃₀FN₇O₇S

¹H NMR (CDCl₃) δ ppm 8.28 (d, 1H), 7.44 (m, 5H), 7.27 (t, 1H), 7.12 (d, 1H), 6.84 (d, 1H), 6.55 (d, 1H), 4.56 (s, 2H), 4.50 (m, 1H), 4.01 (m, 1H), 3.91 (m, 1H), 3.80 (s, 2H), 2.42 (brs, 1H), 2.00 (m 3H), 1.70 (m, 3H), 1.37 (s, 1H), 1.18 (m, 5H).

### Intermediate 29

### Trans-N-(1-(4-aminocyclohexyl)-2-(7-fluoro-2-oxo-1,5-naphthyridin-1(2H)-yl)ethyl)-2-nitrobenzenesulfonamide, trifluoroacetic acid salt, enantiomer A

Trans-tert-Butyl 4-(2-(7-fluoro-2-oxo-1,5-naphthyridin-1(2H)-yl)-1-(2-nitrophenyl-sulfonamido)ethyl)cyclohexylcarbamate, enantiomer A (Intermediate 30, 135 mg, 0.23 mmol) was dissolved in dichloromethane (5 mL) and treated with trifluoroacetic acid (1 mL). The mixture was stirred at room temperature for 2 hours, then concentrated and codistilled with methanol twice under reduced pressure to give 140 mg of the crude product as a single enantiomer.

MS (ES): 490 (MH⁺) for C₂₂H₂₄FN₅O₅

### Intermediate 30

### Trans-tert-butyl 4-(2-(7-fluoro-2-oxo-1,5-naphthyridin-1(2H)-yl)-1-(2-nitrophenylsulfonamido)ethyl)cyclohexylcarbamate, enantiomer A

7-Fluoro-1,5-naphthyridin-2(1H)-one (prepared according to the procedure in WO07138974, 137 mg, 0.83 mmol) was suspended in dry DMF (1 mL) under nitrogen and cooled in an ice bath. Sodium hydride (40 mg, 1.0 mmol) was added and the reaction was removed from the ice bath, stirred at room temperature for 15 minutes. Trans-tert-butyl 4-(1-(2-nitrophenylsulfonyl)-aziridin-2-yl)-cyclohexylcarbamate, ((−) enantiomer (Intermediate 11(b), 355 mg, 0.83 mmol) in 1.0 mL dry DMF was added dropwise. After 30 minutes the mixture was cooled in an ice bath and quenched with potassium phosphate buffer (1M, pH 7), diluted with water and extracted with ethyl acetate twice. The combined organic phases were washed with water (3 times 5 mL), then with brine, dried over sodium sulfate and concentrated under reduced pressure. Chromatography was done on silica gel with 20-100% ethyl acetate in hexanes giving 135 mg of product as a single enantiomer in the form of a yellow foam (27%).

MS (ES): 612 (MH⁺) for C₂₇H₃₂FN₅O₇S

¹H NMR (CDCl₃) δ ppm 8.25 (s, 1H), 7.96 (s, 1H), 7.53 (m, 4H), 7.43 (t, 1H), 7.31 (t, 1H), 6.60 (d, 1H), 6.38 (bs, 1H), 4.56 (d, 1H), 4.39 (m, 1H), 4.12 (m, 1H), 3.88 (m, 1H), 3.26 (brs, 1H), 1.75 (m, 2H), 1.58 (m, 1H), 1.36 (brs, 10H), 1.06 (m, 3H).

### Intermediate 31

### Trans-tert-butyl 4-(2-(7-fluoro-4-methyl-2-oxoquinolin-1(2H)-yl)-1-(2-nitrophenylsulfonamido)ethyl)cyclohexylcarbamate, enantiomer A

To a solution of 7-fluoro-4-methylquinolin-2(1H)-one (prepared according to the procedure described in PCT Pub. No. WO05/066132; 354 mg, 2.00 mmol) in DMF (10 mL) was added sodium hydride (120 mg, 3.0 mmol), and the mixture was stirred at room temperature for 15 minutes under nitrogen. A solution of trans-tert-butyl 4-(1-(2-nitrophenylsulfonyl)aziridin-2-yl)cyclohexylcarbamate, (−) enantiomer (Intermediate 11(b), 850 mg, 2.00 mmol) in DMF (6 mL) was added and the mixture was stirred at room temperature over night. The mixture was quenched with ice water and extracted with ethyl acetate (100 mL). The organic phase was washed with brine twice (2×100 mL) and dried over magnesium sulfate, then concentrated under reduced pressure. Chromatography was done on silica gel with hexanes/acetone 2:1 to give 640 mg (53%) of the title product as a single enantiomer in the form of a colorless solid.

MS (ES): 601 (M−H⁻) for C₂₉H₃₅FN₄O₇S

¹H NMR (DMSO-d₆) δ: 7.40-7.75 (m, 7H); 6.97 (t, 1H); 6.68 (d, 1H); 6.29 (s, 1H); 4.00-4.30 (m, 2H); 3.75 (s, br, 1H); 3.16 (m, 1H); 2.27 (s, 3H); 1.82 (m, 4H); 1.50 (m, 1H); 1.38 (s, 9H); 1.00-1.25 (m, 4H).

### Intermediate 32

### Trans-N-(1-(4-aminocyclohexyl)-2-(7-fluoro-4-methyl-2-oxoquinolin-1(2H)-yl)ethyl)-2-nitrobenzenesulfonamide, enantiomer A, trifluoroacetic acid salt

A solution of trans-tert-butyl 4-(2-(7-fluoro-4-methyl-2-oxoquinolin-1(2H)-yl)-1-(2-nitrophenylsulfonamido)ethyl)cyclohexylcarbamate, enantiomer A (Intermediate 31, 640 mg, 1.06 mmol) in dichloromethane (2 ml) at room temperature was treated with 1/1 dichloromethane/trifluoroacetic acid (2 mL). The mixture was stirred at room temperature for 2 hours. The mixture was concentrated under reduced pressure, then co-distilled with methanol (twice) to give the title product as a single enantiomer in the form of a solid trifluoroacetic acid salt (740 mg).

MS (ES): 503 (MH⁺) for C₂₄H₂₇FN₄O₅S

### Intermediate 33

### Trans-N-(2-(7-fluoro-4-methyl-2-oxoquinolin-1(2H)-yl)-1-(4-((3-oxo-3,4-dihydro-2H-pyrido[3,2-b][1,4]oxazin-6-yl)methylamino)cyclohexyl)ethyl-2-nitrobenzenesulfonamide, enantiomer A

Trans-N-(1-(4-aminocyclohexyl)-2-(7-fluoro-2-oxoquinoxalin-1(2H)-yl)ethyl)-2-nitrobenzenesulfonamide, enantiomer A, trifluoroacetic acid salt (Intermediate 32, 620 mg, 0.99 mmol) was dissolved in DMF (3 mL). Triethylamine (1 mL, 0.71 mmol) and freshly activated MS 3 Å (perled) were added under nitrogen, followed by addition of 3-oxo-3,4-dihydro-2H-pyrido[3,2-b][1,4]oxazine-6-carbaldehyde (prepared according to the procedure described in PCT Pub. No. WO04/058144, 41.9 mg, 0.24 mmol) and the mixture was heated to 80° C. for 12 hours. Sodium triacetoxyborohydride (550 mg, 2.6 mmol) was added and the mixture was stirred at room temperature over night. More sodium cyanoborohydride (122 mg, 1.94 mmol) was added, and the mixture was stirred overght. Saturated sodium bicarbonate solution (100 ml) was added, the mixture was filtered through diatomaceous earth (Celite® brand), the filter cake was washed with methanol. The combined filtrate and wash were concentrated under reduced pressure to an oil. The residue was taken up in dichloromethane (100 ml) and was washed with brine (2×100 ml). The combined organic phases were dried over magnesium sulfate, then concentrated under reduced pressure. Chromatography was done on silica gel, eluting with CH₂Cl₂/MeOH 20:1. Fractions containing product were pooled and concentrated under reduced pressure to give 237 mg (28.9%) of the title product as a single enantiomer in the form of a white foam.

MS (ES): 665 (MH⁺) for C₃₂H₃₃FN₆O₇S

¹H NMR (DMSO-d₆) δ: 11.15 (s, br, 1H); 7.25-7.60 (m, 7H); 6.90-7.05 (m, 2H); 6.28 (s, 1H); 4.61 (s, 2H); 4.24 (m, 1H); 4.07 (m, 1H); 3.75 (m, 3H); 3.17 (d, 1H); 2.33 (m, 1H); 2.26 (m, 3H); 1.93 (m, 4H); 1.51 (m, 1H); 1.05 (m, 4H).

### Example 1

### Trans-6-((4-(1-amino-2-(7-methoxy-2-oxo-1,5-naphthyridin-1(2H)-yl)ethyl)cyclohexylamino)methyl)-2H-pyrido[3,2-b][1,4]oxazin-3(4H)-one

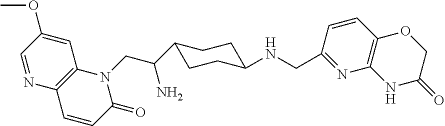

A mixture of crude trans-N-(4-(1-amino-2-(7-methoxy-2-oxo-1,5-naphthyridin-1(2H)-yl)ethyl)cyclohexyl)-2,2,2-trifluoro-N-((3-oxo-3,4-dihydro-2H-pyrido[3,2-b][1,4]oxazin-6-yl)methyl)acetamide (Intermediate 10, 41 mg, 0.07 mmol) in methanol (4 mL) and water (1.00 mL) was heated at 50° C. under stirring for 60 minutes. The mixture was concentrated under reduced pressure and the residue was purified by reverse phase HPLC with acetonitrile/water using ammonium hydroxide as a pH modifier to give the title product as a racemic mixture in the form of a colourless solid, 7.2 mg (21%).

MS (ES): 479 (MH⁺) for C₂₅H₃₀N₆O₄

¹H NMR (DMSO-d₆) δ: 11.5 (s, br, 1H); 8.28 (d, 1H); 7.88 (d, 1H); 7.38 (d, 1H); 7.33 (d, 1H); 7.05 (d, 1H); 6.68 (d, 1H); 4.62 (s, 2H); 4.28 (m, 2H); 3.99 (m, 1H); 3.97 (s, 3H); 3.78 (s, 2H); 3.00 (s, br, 1H); 2.49 (m, 1H); 1.99 (m, 3H); 1.22 (m, 1H); 0.8-1.4 (m, 5H).

The compound of Example 1 was also be prepared according to the following procedure:

### Example 1

### Alternative Synthesis

### Trans-6-((4-(1-amino-2-(7-methoxy-2-oxo-1,5-naphthyridin-1(2H)-yl)ethyl)cyclohexylamino)methyl)-2H-pyrido[3,2-b][1,4]oxazin-3(4H)-one

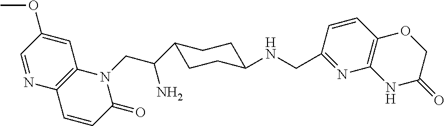

Trans-N-(2-(7-methoxy-2-oxo-1,5-naphthyridin-1(2H)-yl)-1-(4-((3-oxo-3,4-dihydro-2H-pyrido[3,2-b][1,4]oxazin-6-yl)methylamino)-cyclohexyl)ethyl)-2-nitrobenzenesulfonamide (Intermediate 14, 550 mg, 0.83 mmol), benzenethiol (425 μL, 4.14 mmol), and potassium carbonate (572 mg, 4.14 mmol) were reacted using a procedure similar to the one described for the synthesis of Example 2 to give 255 mg (64%) of the title product as a racemic mixture.

MS (ES): 479 (MH⁺) for C₂₅H₃₀N₆O₄

¹H NMR (DMSO-d₆) δ ppm 0.96-1.32 (m, 6H); 1.61-1.71 (m, 1H); 1.84-2.02 (m, 4H); 2.24-2.40 (m, 2H); 2.79-2.89 (m, 1H); 3.70 (s, 2H); 3.96 (s, 3H); 4.12-4.31 (m, 2H); 4.61 (s, 2H); 6.65 (d, 1H); 7.02 (d, 1H); 7.29 (d, 1H); 7.43 (s, 1H); 7.85 (d, 1H); 8.27 (d, 1H); 11.12 (brs, 1H).

The R and S enantiomers of the title product were separated by chiral HPLC (Chiralcel OJ column, 20×250 mm, 10μ, 40% 1:1 methanol:ethanol, 60% hexanes, 10 mL/min) to give Example 1(a) and Example 1(b) as off-white solids.

### Example 1(a)

### First Eluting Compound

### Trans-6-((4-(1-amino-2-(7-methoxy-2-oxo-1,5-naphthyridin-1(2H)-yl)ethyl)cyclohexylamino)methyl)-2H-pyrido[3,2-b][1,4]oxazin-3(4H)-one, enantiomer A

Yield: 84 mg

¹H NMR (DMSO-d₆) δ ppm 0.96-1.32 (m, 6H); 1.61-1.71 (m, 1H); 1.84-2.02 (m, 4H); 2.24-2.40 (m, 2H); 2.79-2.89 (m, 1H); 3.70 (s, 2H); 3.96 (s, 3H); 4.12-4.31 (m, 2H); 4.61 (s, 2H); 6.65 (d, 1H); 7.02 (d, 1H); 7.29 (d, 1H); 7.43 (s, 1H); 7.85 (d, 1H); 8.27 (d, 1H); 11.12 (brs, 1H).

### Example 1(b)

### Second Eluting Compound

### Trans-6-((4-(1-amino-2-(7-methoxy-2-oxo-1,5-naphthyridin-1(2H)-yl)ethyl)cyclohexylamino)methyl)-2H-pyrido[3,2-b][1,4]oxazin-3(4H)-one, enantiomer B

Yield: 82 mg

¹H NMR (DMSO-d₆) δ ppm 0.96-1.32 (m, 6H); 1.61-1.71 (m, 1H); 1.84-2.02 (m, 4H); 2.24-2.40 (m, 2H); 2.79-2.89 (m, 1H); 3.70 (s, 2H); 3.96 (s, 3H); 4.12-4.31 (m, 2H); 4.61 (s, 2H); 6.65 (d, 1H); 7.02 (d, 1H); 7.29 (d, 1H); 7.43 (s, 1H); 7.85 (d, 1H); 8.27 (d, 1H); 11.12 (brs, 1H).

The compound of Example 1(a) was also obtained via a chiral synthesis:

### Example 1(a)

### Alternative Synthesis

### Trans-6-((4-(1-amino-2-(7-methoxy-2-oxo-1,5-naphthyridin-1(2H)-yl)ethyl)cyclohexylamino)methyl)-2H-pyrido[3,2-b][1,4]oxazin-3(4H)-one, enantiomer A

The title product was obtained by following the procedure used for the synthesis of Example 1 (Alternative Synthesis), except instead of Intermediate 14 (racemic mixture), Intermediate 14(a) (single enantiomer) was used as the starting material.

### Example 2

### Trans-6-((4-(1-amino-2-(7-methoxy-2-oxoquinoxalin-1(2H)-yl)ethyl)cyclohexylamino)methyl)-2H-pyrido[3,2-b][1,4]oxazin-3(4H)-one

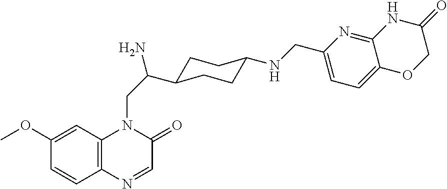

To a solution of trans-N-(2-(7-methoxy-2-oxoquinoxalin-1(2H)-yl)-1-(4-((3-oxo-3,4-dihydro-2H-pyrido[3,2-b][1,4]oxazin-6-yl)methylamino)-cyclohexyl)ethyl)-2-nitrobenzenesulfonamide (Intermediate 17, 140 mg, 0.21 mmol) in DMF (5 mL) was added benzenethiol (108 μL, 1.05 mmol) followed by potassium carbonate (146 mg, 1.05 mmol). The reaction was stirred at room temperature for one hour. Solvent was removed in vacuo. The crude product was partitioned between 15% methanol in dichloromethane and saturated sodium bicarbonate solution. The aqueous phase was re-extracted twice with 15% methanol in dichloromethane and the combined organic phases were dried over sodium sulfate, filtered, and concentrated in vacuo. Chromatography was done on silica gel with a gradient of 5-15% methanol in dichloromethane containing 0.25% ammonium hydroxide to give 58 mg (58%) of the title product as a racemic mixture.

MS (ES): 479 (MH⁺) for C₂₅H₃₀N₆O₄

¹H NMR (DMSO-d₆) 6: ppm 0.94-1.35 (m, 6H); 1.62-1.74 (m, 1H); 1.84-2.03 (m, 4H); 2.23-2.40 (m, 2H); 2.90 (brs, 1H); 3.71 (s, 2H); 3.90 (s, 3H); 4.17 (d, 2H); 4.61 (s, 2H); 6.95-7.06 (m, 3H); 7.30 (d, 1H); 7.74 (d, 1H); 8.03 (s, 1H); 11.15 (brs, 1H).

The R and S enantiomers of the title product were separated by chiral HPLC (Chiralpak® IB column, 20×250 mm, 5μ, 20% 1:1 methanol:ethanol, 80% hexanes, 20 mL/min) to give Example 2(a) and Example 2(b) as off-white solids:

### Example 2(a)

### First Eluting Compound

### Trans-6-((4-(1-amino-2-(7-methoxy-2-oxoquinoxalin-1(2H)-yl)ethyl)cyclohexylamino)methyl)-2H-pyrido[3,2-b][1,4]oxazin-3(4H)-one, enantiomer A

Yield: 20 mg

¹H NMR (DMSO-d₆) 6: ppm 0.94-1.35 (m, 6H); 1.62-1.74 (m, 1H); 1.84-2.03 (m, 4H); 2.23-2.40 (m, 2H); 2.90 (brs, 1H); 3.71 (s, 2H); 3.90 (s, 3H); 4.17 (d, 2H); 4.61 (s, 2H); 6.95-7.06 (m, 3H); 7.30 (d, 1H); 7.74 (d, 1H); 8.03 (s, 1H); 11.15 (brs, 1H).

### Example 2(b)

### Second Eluting Compound

### Trans-6-((4-(1-amino-2-(7-methoxy-2-oxoquinoxalin-1(2H)-yl)ethyl)cyclohexylamino)methyl)-2H-pyrido[3,2-b][1,4]oxazin-3(4H)-one, enantiomer B

Yield: 20 mg

¹H NMR (DMSO-d₆) δ: ppm 0.94-1.35 (m, 6H); 1.62-1.74 (m, 1H); 1.84-2.03 (m, 4H); 2.23-2.40 (m, 2H); 2.90 (brs, 1H); 3.71 (s, 2H); 3.90 (s, 3H); 4.17 (d, 2H); 4.61 (s, 2H); 6.95-7.06 (m, 3H); 7.30 (d, 1H); 7.74 (d, 1H); 8.03 (s, 1H); 11.15 (brs, 1H).

The compound of Example 2(b) was also obtained via a chiral synthesis:

### Example 2(b)

### Alternative Synthesis

### Trans-6-((4-(1-Amino-2-(7-methoxy-2-oxoquinoxalin-1(2H)-yl)ethyl)cyclohexylamino)methyl)-2H-pyrido[3,2-b][1,4]oxazin-3(4H)-one, enantiomer B

The title product was obtained by following the procedure used for the synthesis of Example 2 (racemic mixture), except that instead of Intermediate 17 (racemic mixture), Intermediate 17(a) (single enantiomer) was used as the starting material.

### Example 3

### Trans-2-((4-(1-Amino-2-(7-methoxy-2-oxoquinoxalin-1(2H)-yl)ethyl)cyclohexylamino)methyl)-6H-pyrimido[5,4-b][1,4]oxazin-7(8H)-one

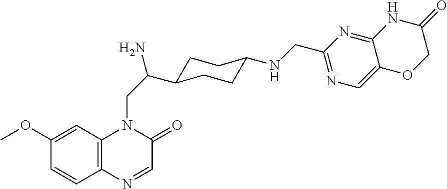

Trans-N-(2-(7-methoxy-2-oxoquinoxalin-1(2H)-yl)-1-(4-((7-oxo-7,8-dihydro-6H-pyrimido[5,4-b][1,4]oxazin-2-yl)methylamino)-cyclohexyl)ethyl)-2-nitrobenzenesulfonamide (Intermediate 18, 850 mg, 1.28 mmol), benzenethiol (657 μL, 6.39 mmol), and potassium carbonate (882, 6.39 mmol) were reacted using a procedure similar to the one described for the synthesis of Example 2 to give 276 mg (45%) of the title product as a racemic mixture.

MS (ES): 480 (MH⁺) for C₂₄H₂₉N₇O₄

¹H NMR (DMSO-d₆) δ: ppm 0.95-1.36 (m, 6H); 1.62-1.75 (m, 1H); 1.83-2.03 (m, 4H); 2.29-2.46 (m, 2H); 2.91 (brs, 1H); 3.78 (s, 2H); 3.90 (s, 3H); 4.12-4.23 (m, 2H); 4.73 (s, 2H); 6.93-7.04 (m, 2H); 7.74 (d, 1H); 8.04 (s, 1H); 8.24 (s, 1H).

The R and S enantiomers of the title product were separated by chiral HPLC (Chiralpak® IB column, 20×250 mm, 5μ, 40% 1:1 methanol:ethanol, 60% hexanes, 20 mL/min) to give Example 3(a) and Example 3(b) as off-white solids:

### Example 3(a)

### First Eluting Compound

### Trans-2-((4-(1-amino-2-(7-methoxy-2-oxoquinoxalin-1(2H)-yl)ethyl)cyclohexylamino)methyl)-6H-pyrimido[5,4-b][1,4]oxazin-7(8H)-one, enantiomer A

Yield: 70 mg

¹H NMR (DMSO-d₆) 6: ppm 0.95-1.36 (m, 6H); 1.62-1.75 (m, 1H); 1.83-2.03 (m, 4H); 2.29-2.46 (m, 2H); 2.91 (brs, 1H); 3.78 (s, 2H); 3.90 (s, 3H); 4.12-4.23 (m, 2H); 4.73 (s, 2H); 6.93-7.04 (m, 2H); 7.74 (d, 1H); 8.04 (s, 1H); 8.24 (s, 1H).

### Example 3(b)

### Second Eluting Compound

### Trans-2-((4-(1-amino-2-(7-methoxy-2-oxoquinoxalin-1(2H)-yl)ethyl)cyclohexylamino)methyl)-6H-pyrimido[5,4-b][1,4]oxazin-7(8H)-one, enantiomer B

Yield: 67 mg

¹H NMR (DMSO-d₆) δ: ppm 0.95-1.36 (m, 6H); 1.62-1.75 (m, 1H); 1.83-2.03 (m, 4H); 2.29-2.46 (m, 2H); 2.91 (brs, 1H); 3.78 (s, 2H); 3.90 (s, 3H); 4.12-4.23 (m, 2H); 4.73 (s, 2H); 6.93-7.04 (m, 2H); 7.74 (d, 1H); 8.04 (s, 1H); 8.24 (s, 1H).

The compound of Example 3(b) was also obtained via a chiral synthesis:

### Example 3(b)

### Alternative Synthesis

### Trans-2-((4-(1-amino-2-(7-methoxy-2-oxoquinoxalin-1(2H)-yl)ethyl)cyclohexylamino)methyl)-6H-pyrimido[5,4-b][1,4]oxazin-7(8H)-one, enantiomer B

The title product was obtained by following the procedure used for the synthesis of Example 3 (racemic mixture), except that instead of Intermediate 18 (racemic mixture), Intermediate 18(a) (single enantiomer) was used as the starting material.

### Example 4

### Trans-1-(2-amino-2-(4-((3-oxo-3,4-dihydro-2H-pyrido[3,2-b][1,4]oxazin-6-yl)methylamino)cyclohexyl)ethyl)-2-oxo-1,2-dihydroquinoline-7-carbonitrile

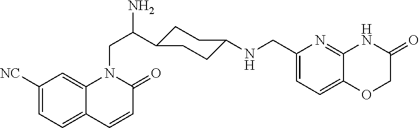

Trans-N-(2-(7-cyano-2-oxoquinolin-1(2H)-yl)-1-(4-((3-oxo-3,4-dihydro-2H-pyrido[3,2-b][1,4]oxazin-6-yl)methylamino)-cyclohexyl)ethyl)-2-nitrobenzenesulfonamide (Intermediate 21, 0.563 g, 0.86 mmol), benzenethiol (0.879 mL, 8.56 mmol), and potassium carbonate (1.183 g, 8.56 mmol) were reacted using a procedure similar to the one described for the synthesis of Example 2 to give 212 mg (52%) of the title product as a racemic mixture.

MS (ES): 473 (MH⁺) for C₂₆H₂₈N₆O₃

¹H NMR (DMSO-d₆) δ: 0.94-1.14 (m, 4H); 1.26 (m, 2H); 1.60 (m, 2H); 1.93 (m, 4H); 2.31 (m, 1H); 2.83 (m, 1H); 3.70 (s, 2H); 4.13 (m, 1H); 4.30 (m, 1H); 4.61 (s, 2H); 6.78 (d, 1H); 7.03 (d, 1H); 7.30 (d, 1H); 7.63 (d, 1H); 7.90 (d, 1H); 7.99 (d, 1H); 8.18 (s, 1H).

The R and S enantiomers of the title product were separated by chiral HPLC (Chiralpak® IB, 70% hexane, 30% 1:1 EtOH:MeOH, 0.1% DEA) to give Example 4(a) and Example 4(b) as yellow foams:

### Example 4(a)

### First Eluting Compound

### Trans-1-(2-amino-2-(4-((3-oxo-3,4-dihydro-2H-pyrido[3,2-b][1,4]oxazin-6-yl)methylamino)cyclohexyl)ethyl)-2-oxo-1,2-dihydroquinoline-7-carbonitrile, enantiomer A

Yield: 83 mg

¹H NMR (DMSO-d₆) δ: 0.94-1.14 (m, 4H); 1.26 (m, 2H); 1.60 (m, 2H); 1.93 (m, 4H); 2.31 (m, 1H); 2.83 (m, 1H); 3.70 (s, 2H); 4.13 (m, 1H); 4.30 (m, 1H); 4.61 (s, 2H); 6.78 (d, 1H); 7.03 (d, 1H); 7.30 (d, 1H); 7.63 (d, 1H); 7.90 (d, 1H); 7.99 (d, 1H); 8.18 (s, 1H).

### Example 4(b)

### Second Eluting Compound

### Trans-1-(2-amino-2-(4-((3-oxo-3,4-dihydro-2H-pyrido[3,2-b][1,4]oxazin-6-yl)methylamino)cyclohexyl)ethyl)-2-oxo-1,2-dihydroquinoline-7-carbonitrile, enantiomer B

Yield: 82 mg

¹H NMR (DMSO-d₆) δ: 0.94-1.14 (m, 4H); 1.26 (m, 2H); 1.60 (m, 2H); 1.93 (m, 4H); 2.31 (m, 1H); 2.83 (m, 1H); 3.70 (s, 2H); 4.13 (m, 1H); 4.30 (m, 1H); 4.61 (s, 2H); 6.78 (d, 1H); 7.03 (d, 1H); 7.30 (d, 1H); 7.63 (d, 1H); 7.90 (d, 1H); 7.99 (d, 1H); 8.18 (s, 1H).

### Example 5

### Trans-6-((4-(1-amino-2-(7-fluoro-2-oxoquinoxalin-1(2H)-yl)ethyl)cyclohexylamino)methyl-2H-pyrido[3,2-b][1,4]oxazin-3(4H)-one, (+) enantiomer

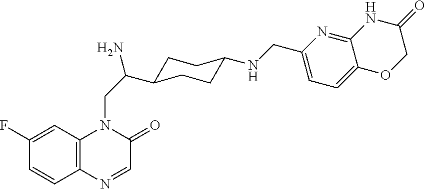

A solution of trans-N-(2-(7-fluoro-2-oxoquinoxalin-1(2H)-yl)-1-(4-((3-oxo-3,4-dihydro-2H-pyrido[3,2-b][1,4]oxazin-6-yl)methylamino)cyclohexyl)ethyl)-2-nitrobenzenesulfonamide, enantiomer A (Intermediate 22, 121 mg, 0.19 mmol) in DMF (3 mL) was treated with benzenethiol (0.096 mL, 0.93 mmol) followed by potassium carbonate (128 mg, 0.93 mmol) and the mixture was vigorously stirred over night. More and potassium carbonate (64 mg) and benzenethiol (48 uL) were added and the mixture was stirred over night. Acetic acid (0.159 mL, 2.79 mmol) was added and the mixture was stirred for 10 minutes. The mixture was concentrated by codistillation with toluene. The residue was taken up in dichloromethane (˜30 mL) and saturated aqueous sodium bicarbonate solution (3 mL). Water (2 mL) was added and the pH was adjusted to ˜10 with 15% NaOH. The organic phase was separated and the aqueous phase was backextracted with dichloromethane (4 times 30 mL). The combined organic phases were dried over sodium sulfate, concentrated under reduced pressure, and the residue was codistilled with toluene twice. Chromatography was done on silica gel with dichloromethane/methanol 20:1 to 10:1, containing 0.25% ammonium hydroxide. Fractions containing product were pooled and concentrated under reduced pressure. The residue was codistilled with methanol twice, then with dichloromethane twice. The residue was taken up in dichloromethane (˜3 mL) and triturated with ether (5 mL) over night. The solid was collected by filtration. The mother liquors were concentrated and the residue was taken up in hot ethyl acetate (˜2 mL) and hexanes (˜2 mL) were added and the mixture was left at room temperature for 1 hour, then the solid was collected by filtration. The solids were combined to give 28.3 mg of the title product as a slightly yellow solid.

Optical rotation: [α]_{D}=+31.9 (c=1, methanol).

MS (ES): 467 (MH⁺) for C₂₄H₂₇FN₆O₃

¹H NMR (DMSO-d₆) δ: ppm 11.16 (brs, 1H); 8.18 (s, 1H); 7.86 (dd, 1H); 7.56 (dd, 1H); 7.30 (d, 1H); 7.22 (dd, 1H); 7.03 (d, 1H); 4.61 (s, 2H); 4.21-4.05 (m, 2H); 3.72 (s, 2H); 2.88 (m, 1H); 2.35 (m, 1H); 2.02-1.83 (m, 4H); 1.64 (m, 1H); 1.35-0.93 (m, 4H).

### Example 6

### Trans-2-((4-(1-amino-2-(7-fluoro-2-oxoquinoxalin-1(2H)-yl)ethyl)cyclohexylamino)methyl-6H-pyrimido[5,4-b][1,4]oxazin-7(8H)-one, (+) enantiomer

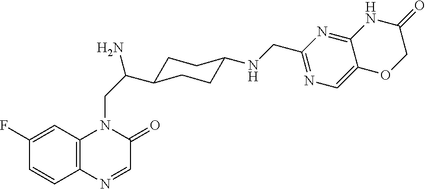

Trans-N-(2-(7-fluoro-2-oxoquinoxalin-1(2H)-yl)-1-(4-((7-oxo-7,8-dihydro-6H-pyrimido[5,4-b][1,4]oxazin-2-yl)methylamino)cyclohexyl)ethyl)-2-nitrobenzenesulfonamide, enantiomer A (Intermediate 25, 103 mg, 0.16 mmol) was reacted with benzenethiol (0.162 mL, 1.58 mmol) and potassium carbonate (218 mg, 1.58 mmol) as described for Example 5 to give 30 mg of the title product as a slightly yellow solid (optical rotation: [α]_{D}=+34.8 (c=1, methanol)).

MS (ES): 468 (MH⁺) for C₂₃H₂₆FN₇O₃

¹H NMR (DMSO-d₆) 6: ppm 8.25 (s, 1H); 8.18 (s, 1H); 7.86 (dd, 1H); 7.56 (m, 1H); 7.23 (ddd, 1H); 4.73 (s, 2H); 4.13 (m, 2H); 3.78 (s, 2H); 2.86 (m, 1H); 2.37 (m, 1H); 2.02-1.82 (m, 4H); 1.64 (m, 1H); 1.38-0.95 (4H).

### Example 7

### Trans-1-(2-amino-2-(4-((3-oxo-3,4-dihydro-2H-pyrido[3,2-b][1,4]thiazin-6-yl)methylamino)cyclohexyl)ethyl)-2-oxo-1,2-dihydroquinoline-7-carbonitrile

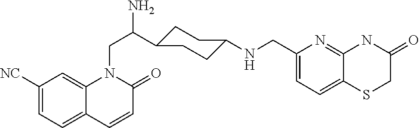

Trans-N-(2-(7-cyano-2-oxoquinolin-1(2H)-yl)-1-(4-((3-oxo-3,4-dihydro-2H-pyrido[3,2-b][1,4]thiazin-6-yl)methylamino)-cyclohexyl)ethyl)-2-nitrobenzenesulfonamide (Intermediate 26, 0.439 g, 0.65 mmol), benzenethiol (0.669 mL, 6.52 mmol), and potassium carbonate (0.90 g, 6.52 mmol) were reacted as described for Examples 2 and 3 to give 127 mg (40%) of a racemic mixture of the title product.

MS (ES): 489 (MH⁺) for C₂₆H₂₈N₆O₂S

¹H NMR (DMSO-d₆) δ: 0.95-1.14 (m, 4H); 1.26 (m, 2H); 1.59 (m, 2H); 1.93 (m, 4H); 2.30 (m, 1H); 2.81 (m, 1H); 3.52 (s, 2H); 3.73 (s, 2H); 4.12 (m, 1H); 4.30 (m, 1H); 6.77 (d, 1H); 7.10 (d, 1H); 7.62 (d, 1H); 7.73 (d, 1H); 7.89 (d, 1H); 7.99 (d, 1H); 8.18 (s, 1H); 10.87 (s, 1H).

The R and S enantiomers of the Example 7 were separated by chiral HPLC (Chiralpak® IB, 70% hexane, 30% 1:1 EtOH:MeOH, 0.1% DEA) to give Example 7(a) and Example 7(b) as yellow foams:

### Example 7(a)

### First Eluting Compound

### Trans-1-(2-amino-2-(4-((3-oxo-3,4-dihydro-2H-pyrido[3,2-b][1,4]thiazin-6-yl)methylamino)cyclohexyl)ethyl)-2-oxo-1,2-dihydroquinoline-7-carbonitrile, enantiomer A

43 mg, >98% ee.

### Example 7(b)

### Second Eluting Compound

### Trans-1-(2-amino-2-(4-((3-oxo-3,4-dihydro-2H-pyrido[3,2-b][1,4]thiazin-6-yl)methylamino)cyclohexyl)ethyl)-2-oxo-1,2-dihydroquinoline-7-carbonitrile, enantiomer B

42 mg, 97% ee.

### Example 8

### Trans-2-((4-(1-amino-2-(7-methoxy-2-oxo-1,5-naphthyridin-1(2H)-yl)ethyl)cyclohexylamino)methyl)-6H-pyrimido[5,4-b][1,4]oxazin-7(8H)-one

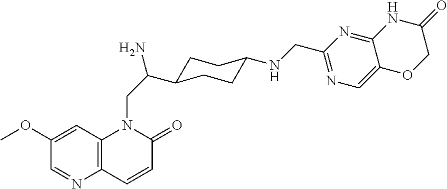

To a solution of trans-N-(2-(7-methoxy-2-oxo-1,5-naphthyridin-1(2H)-yl)-1-(4-((7-oxo-7,8-dihydro-6H-pyrimido[5,4-b][1,4]oxazin-2-yl)methylamino)cyclohexyl)ethyl)-2-nitrobenzenesulfonamide (Intermediate 27, 500 mg, 0.75 mmol) in DMF (10 mL) was added benzenethiol (386 μL, 3.76 mmol) followed by potassium carbonate (519 mg, 3.76 mmol). The reaction was stirred at room temperature for 1 hr. DMF was removed under reduced pressure. The crude product was partitioned between 15% methanol in dichloromethane and saturated sodium bicarbonate solution. The layers were separated and the aqueous phase was re-extracted twice with 15% methanol in dichloromethane. The combined organic phases were dried over sodium sulfate and concentrated under reduced pressure. Chromatography was done on silica gel with a gradient of 5-15% methanol in dichloromethane containing 0.25% ammonium hydroxide to give 124 mg (34%) of the title product as a racemic mixture in the form of a solid.

MS (ES): 480 (MH⁺) for C₂₄H₂₉N₇O₄

¹H NMR (300 DMSO-d₆) δ ppm 1.01-1.49 (m, 6H); 1.65-1.83 (m, 1H); 1.89-2.10 (m, 3H); 3.02 (brs, 1H); 3.86 (s, 2H); 3.98 (s, 3H); 4.20-4.35 (m, 2H); 4.75 (s, 2H); 6.68 (d, 1H); 7.39 (s, 1H); 7.87 (d, 1H); 8.24-8.32 (m, 1H).

### Example 9

### Trans-6-((4-(1-amino-2-(2-oxo-7-(phenylthio)-1,5-naphthyridin-1(2H)-yl)ethyl)cyclohexylamino)methyl)-2H-pyrido[3,2-b][1,4]oxazin-3(4H)-one, enantiomer A

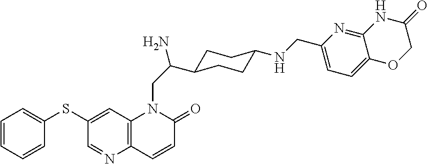

Trans-N-(2-(7-fluoro-2-oxo-1,5-naphthyridin-1(2H)-yl)-1-(4-((3-oxo-3,4-dihydro-2H-pyrido[3,2-b][1,4]oxazin-6-yl)methylamino)cyclohexyl)ethyl)-2-nitrobenzene-sulfonamide, enantiomer A (Intermediate 28, 65 mg, 0.10 mmol) was dissolved in DMF (1 mL), treated with K₂CO₃ (69 mg, 0.50 mmol) followed by benzenethiol (55 mg, 0.50 mmol), and stirred at room temperature overnight. The mixture was partitioned between 15% methanol in dichloromethane and saturated sodium bicarbonate. The organic layer was separated and the aqueous phase was re-extracted once with 15% methanol in dichloromethane. The combined organic layers were dried over sodium sulfate and concentrated under reduced pressure. Chromatography was done on silica gel with 0-10% methanol in dichloromethane, containing 0.25% ammonium hydroxide to give 31 mg of the title product as a colorless film (56%).

MS (ES): 557 (MH⁺) for C₃₀H₃₂N₆O₃S

¹H NMR (CDCl₃) δ ppm 8.32 (d, 1H), 7.78 (d, 1H), 7.46 (m, 2H), 7.36 (m, 4H), 7.13 (d, 2H), 6.88 (d, 1H), 6.76 (d, 1H), 4.55 (s, 2H), 4.33 (dd, 1H), 3.80 (s, 2H), 3.68 (dd, 1H), 3.39 (s, 1H), 2.66 (m, 1H), 2.38 (m, 1H), 1.97 (m, 2H), 1.69 (m, 1H), 1.54 (d, 1H), 1.12 (m, 6H), 0.84 (d, 1H).

### Example 10

Trans-6-((4-(1-amino-2-(7-fluoro-4-methyl-2-oxoquinolin-1(2H)-yl)ethyl)cyclohexylamino)methyl)-2H-pyrido[3,2-b][1,4]oxazin-3(4H)-one, enantiomer A

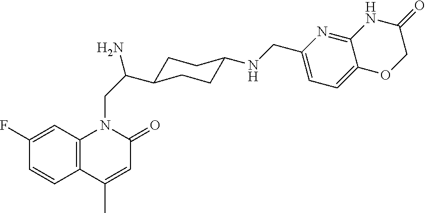

To a solution of trans-N-(2-(7-fluoro-4-methyl-2-oxoquinolin-1(2H)-yl)-1-(4-((3-oxo-3,4-dihydro-2H-pyrido[3,2-b][1,4]oxazin-6-yl)methylamino)cyclohexyl)ethyl)-2-nitrobenzenesulfonamide, enantiomer A (Intermediate 33, 235 mg, 0.35 mmol) in DMF (5.00 mL) was added potassium carbonate (250 mg, 1.81 mmol) and benzenethiol (104.0 mg, 0.94 mmol). The mixture was stirred at room temperature overnight. The reaction mixture was extracted with ethyl acetate (50 mL) and was washed with brine (3×50 mL). The organic layer was dried over magnesium sulfate and a yellow oil was obtained upon removal of solvent at reduced pressure. The crude product was then purified by reverse phase HPLC using water/trifluoroacetic acid (0.1%) with acetonitrile gradient of 5-95%. The collected fractions were combined and concentrated to dryness. The product was redissolved in dichloromethane (100 mL) and basified by a potassium carbonate solution (20 mL) and extracted with dichloromethane (2×100 mL) the organic was dried over magnesium sulfate and 42 mg (24.8%) of white solid was obtained as free base upon removal of solvent at reduced pressure.

MS (ES): 480 (MH⁺) for C₂₆H₃₀FN₅O₃

¹H NMR (300 DMSO-d₆) δ ppm 0.90-1.42 (m, 6H); 1.55 (m, 1H); 1.92 (m, 3H); 2.30 (t, 1H); 2.42 (s, 3H); 2.84 (s, 1H); 3.69 (s, 2H); 4.00-4.40 (m, 2H); 4.60 (s, 2H); 6.48 (s, 1H); 7.02 (d, 1H); 7.13 (t, 1H); 7.28 (d, 1H); 7.44 (d, 1H); 7.81 (t, 1H).

What is claimed is:

1-7. (canceled)

8. A compound which is trans-2-[({4-[(1R)-1-amino-2-(7-fluoro-2-oxoquinoxalin-1(2H)-yl)ethyl]cyclohexyl}amino)methyl]-6H-pyrimido[5,4-b][1,4]oxazin-7(8H)-one, or a pharmaceutically acceptable salt thereof.

9. A method for treating a bacterial infection in a warm-blooded animal such as man, said method comprising administering to said animal an effective amount of trans-2-[({4-[(1R)-1-amino-2-(7-fluoro-2-oxoquinoxalin-1(2H)-yl)ethyl]cyclohexyl}amino)methyl]-6H-pyrimido[5,4-b][1,4]oxazin-7(8H)-one, or a pharmaceutically acceptable salt thereof, as claimed in claim 8.

10. A pharmaceutical composition comprising trans-2-[({4-[(1R)-1-amino-2-(7-fluoro-2-oxoquinoxalin-1(2H)-yl)ethyl]cyclohexyl}amino)methyl]-6H-pyrimido[5,4-b][1,4]oxazin-7(8H)-one, or a pharmaceutically acceptable salt thereof, as claimed in claim 8, and at least one pharmaceutically acceptable carrier, diluent, or excipient.
